SEQUENCE LISTING (3 sequences)

SEQ ID NO: 1 - protein, 7 aa, Artificial
  Artificial peptide
NLLMAAS

SEQ ID NO: 2 - protein, 7 aa, Artificial
  Artificial peptide
HHHRHSF

SEQ ID NO: 3 - protein, 23 aa, Artificial
  Artificial peptide
TTGSSHFLIIGFMRRALCGAGSS

(19) United States
(12) Patent Application Publication
(10) Pub. No.: US 2007/0292396 A1
(43) Pub. Date: Dec. 20, 2007

(54) COMBINATION THERAPY WITH ONCOLYTIC ADENOVIRUS

(76) Inventors: Juan Fueyo, Houston, TX (US); Candelaria Gomez-Manzano, Houston, TX (US); W.K. Alfred Yung, Houston, TX (US); Charles A. Conrad, Spring, TX (US); Frederick F. Lang JR., Houston, TX (US)

Correspondence Address: FULBRIGHT & JAWORSKI L.L.P., 600 CONGRESS AVE., SUITE 2400, AUSTIN, TX, 78701, US

(21) Appl. No.: 11/681,592

(22) Filed: Mar. 2, 2007

Related U.S. Application Data
(60) Provisional application No. 60/778,595, filed on Mar. 2, 2006.

(51) Int. Cl. A01N 63/00; A61K 38/00; A61P 43/00

(52) U.S. Cl. 424/93.6; 514/2

(57) ABSTRACT

The present invention involves compositions and methods for treating cancer using a combination of cell cycle modulating agent(s) and anticancer agents or therapies, particularly S-phase specific therapies.

This application claims the benfit of U.S. Provisional Patent Application No. 60/778,595, filed on Mar. 2, 2006.

### BACKGROUND OF THE INVENTION

1. Field of the Invention

The invention generally relates to the field of oncology and cancer therapy. More particularly, it concerns compositions and methods of treating cancer with a first agent that modulates the cell cycle and a second agent that is an anti-cancer agent.

2. Background

During 2006 approximately 190,000 people in the United States and many more around the world will be diagnosed with a primary or metastatic brain tumor. At present, brain tumors are treated by surgery, radiation therapy and chemotherapy, frequently used in combination. However, the prognosis of patients with gliomas have hardly changed in the last 20 years, and only 30 percent of patients survive five years following the diagnosis of a primary malignant brain tumor. Thus, new effective therapeutic approaches are greatly needed.

### SUMMARY OF THE INVENTION

Embodiments of the invention include methods of treating a subject with cancer comprising: a) administering to a patient an effective amount of a cell cycle modulating agent that elevates the proportion of cancer cells in S-phase of the cell cycle (as exemplified with adenovirus and particular delta-24 containing adenovirus); and b) administering an effective amount of an anti-cancer therapy to a subject in need thereof. Typically the combined action of these two types of agents will provide a benefit that is more than the additive effects of each agent administered as a single agent. A cell cycle modulating agent is an agent that when exposed to a cell increases or delays the transition time from one phase of the cell cycle to another or alters the proportion of time the cell is in one phase of the cell cycle relative to another. In certain aspects, the cell cycle modulating agent will block or slow the transition from one cell cycle phase to another, thus resulting in a greater portion, fraction, proportion, or number cells in a sample or target tissue that are a particular phase or phases of the cell cycle while reducing the number of cells in another phase. In further aspects, the target cells are synchronized or partially synchronized in one or more phase of the cell cycle. One or more cell cycle modulating agent can be administered to a subject before, during, after, or concurrently with administration of one or more anti-cancer therapy. The cell cycle modulating agent and the anti-cancer therapy (collectively agents of the invention) may be administered in 1, 2, 3, 4, or more composition in any combination thereof. A cell cycle modulating agent can be a virus (e.g., adenovirus such as Delta-24), a small molecule, a peptide (in certain aspects peptides that target the E2F1 binding to Rb protein), a small interfering RNAs (such as, but not limited to siRNA Rb, siRNA p16; siRNAp53), an oligonucleotide (such as antisense oligonucleotides against Rb and Rb-related pocket proteins, p16 and any other CDK inhibitors, p53), a ribozyme (including, but to limited to antisense oligonucleotides against Rb and Rb-related pocket proteins, p16 and any other CDK inhibitors, p53), a dominant negative protein that effects cell cycle progression, antibodies directed to components of the cell machinery, and/or nanoparticles. In certain aspects the cell cycle modulator is a virus such as an adenovirus. In yet further aspects of the invention the adenovirus is an oncolytic adenovirus, such as, but not limited to the Delta-24 family of adenovirus, which include ICOVIR-5 virus and its derivatives.

A cell cycle modulator may be associated or operably coupled with a targeting moiety, such as peptide or a liposome that is functionalized with a targeting moiety. Targeting moieties include small molecules, peptides, proteins, antibodies and the like that localize or increase the propensity of an agent to associate with a particular subset of organs, tissue, or cell types. The term operably coupled includes direct and indirectly coupled components or agents as well as covalently attached components or agents. In certain embodiments of the invention an oncolytic adenovirus comprises a targeting moiety. A targeting moiety may comprise a modified fiber protein. A modified fiber protein can include a fiber protein both, physically or genetically modified by operably coupling or inserting a heterologous amino acids sequence to or in the fiber protein. In a further aspect a heterologous amino acid sequence is inserted in the HI loop of the fiber protein. A heterologous amino acid sequences include, but are not limited to RGD-4C, NLLMAAS (SEQ ID NO:1), HHHRHSF (SEQ ID NO2), TTGSSHFLIIGFMRRALCGAGSS (SEQ ID NO:3) or others that are readily identifiable by those of skill in the art.

Embodiments of the invention include cell cycle modulators that include oncolytic adenovirus having a decreased E1A mediated toxicity. E1A mediated toxicity can be reduced by modulation of E1A expression. In certain aspects, modulation of E1A expression is effected by substitution of the E1A promoter with a heterologous promoter and/or genetically insulating the E1A promoter from adenoviral enhancers or promoter. An example of such a heterologous promoter is E2F1 promoter. A heterologous promoter may comprise two, four, six or eight E2F1 promoter sequences. In certain embodiments, at least two, four, six, or eight E2F1 promoter sequences are preceded by one or more insulator sequences. An insulator sequence is a genetic element(s) that represses or reduces enhancer-promoter interactions. An example of an insulator element is the human myotonic dystrophy (DM-1) insulator genomic DNA from nt 13006 to nt 13474 of DM-1 locus (GenBank accession no. L08835). This region contains the CTCF-binding sites and the CTG repeats responsible for the insulator activity of the DM-1 locus. A variety of insulator sequences will be readily identifiable by one of skill in the art.

In certain embodiments of the invention an anti-cancer therapy (or agent) is radiation therapy, chemotherapy, immunotherapy, gene therapy, or anti-angiogenic therapy. In certain aspects of the methods described the anti-cancer therapy is chemotherapy. In a further aspect the chemotherapy is at the S-phase of the cell cycle. Thus, in certain preferred aspects, the chemotherapeutic agent is an agent that more cytotoxic to cells in S-phase relative to cells in other phases of the cell cycle. The chemotherapy can be an antimetabolite, a topoisomerase I inhibitor, a topoisomerase II inhibitor, or other agent(s) that complement the cell cycle modulating agent of the invention. A topoisomerse I inhibitor can be CPT11. A variety of know chemotherapy agents or protocols may be used including, but not limited to Topoisomerase I inhibitors [CPT-11 (irinotecan), camptothecin, topotecan]; Topoisomerase II inhibitors (doxorubicin, daunorubicin); Alkalators (temozolomide, carmustine, lomustine, dacarbazine, DTIC, cytoxin, procarbazine); Inhibitors of PKC and/or CDKs: Flavopiridol, Staurosporine, UCN-01, Paullones, Indirubins, Roscovitine, Purvalanol; Inhibitors of Farnesyltransferase: [ZARNESTRA™ (R115777), Sarazar (SCH66336)]; Inhibitors of histone deacetylase: BMS-214662, Trichostatin A, Trapoxin, MS-27-275, FR901228; Inhibitors of HMG-CoA: Mevastatin, Lovastatin; Inhibitors Cdk2,4,6: Retinoids, Fenretinide; EGFR tyrosine kinase inhibitors: Iressa (Gefitinib), Tarceva (Erlotinib); Proteasome inhibitor that increases p21/decreases Cdk1: PS-341; Decreases Cdk1: Arsenic trioxide; platinium compounds (carboplatin, cis-platin, oxaloplatin); Anti-angiogenic agents (Avastin, VEGF trap, PTK787, AEE788); antimetabolite (5-fluorouricil, xeloda, methotrexate, Ara-C, depo-Ara-C, 6-thioguanine); Vinca Alkaloids (vincristine, vinblastine); Taxanes (Taxol, Taxotere) PI3K/Akt/mTor inhibitors, and/or RAD001

Typically chemotherapy will comprise an alkylating agent, mitotic inhibitor, antibiotic, or antimetabolite. Other chemotherapy agents or protocols may include temozolomide, epothilones, melphalan, carmustine, busulfan, lomustine, cyclophosphamide, dacarbazine, polifeprosan, ifosfamide, chlorambucil, mechlorethamine, busulfan, cyclophosphamide, carboplatin, cisplatin, thiotepa, capecitabine, streptozocin, bicalutamide, flutamide, nilutamide, leuprolide acetate, doxorubicin hydrochloride, bleomycin sulfate, daunorubicin hydrochloride, dactinomycin, liposomal daunorubicin citrate, liposomal doxorubicin hydrochloride, epirubicin hydrochloride, idarubicin hydrochloride, mitomycin, doxorubicin, valrubicin, anastrozole, toremifene citrate, cytarabine, fluorouracil, fludarabine, floxuridine, interferon α-2b, plicamycin, mercaptopurine, methotrexate, interferon α-2a, medroxyprogersterone acetate, estramustine phosphate sodium, estradiol, leuprolide acetate, megestrol acetate, octreotide acetate, deithylstilbestrol diphosphate, testolactone, goserelin acetate, etoposide phosphate, vincristine sulfate, etoposide, vinblastine, etoposide, vincristine sulfate, teniposide, trastuzumab, gemtuzumab ozogamicin, rituximab, exemestane, irinotecan hydrocholride, asparaginase, gemcitabine hydrochloride, altretamine, topotecan hydrochloride, hydroxyurea, cladribine, mitotane, procarbazine hydrochloride, vinorelbine tartrate, pentrostatin sodium, mitoxantrone, pegaspargase, denileukin difitix, altretinoin, porfimer, bexarotene, paclitaxel, docetaxel, arsenic trioxide, tretinoin or a number of combinations or formulations thereof. In particular embodiments the chemotherapy is CPT-11, temozolomide, or a platin compound. Radiation therapy can comprise X-ray irradiation, UV-irradiation, y-irradiation, or microwaves.

In still further embodiments of the invention, methods can further comprise subjecting the subject to surgical therapy. One or more of the cell cycle modulating agent, the anti-cancer therapy, or both the cell cycle modulating agent and the anti-cancer therapy can be administered established medical route of administration, such as but not limited to intravenous, intratumoral, or intracranial administration. The agents of the invention may be administered systemically or locally, or both systemically and locally to a subject. In certain aspects the cell cycle modulating agent, the anti-cancer therapy, or both the cell cycle modulating agent and the anti-cancer therapy are administered at least intracranially and/or intratumorally. The cell cycle modulating agent, the anti-cancer therapy, or both the cell cycle modulating agent and the anti-cancer therapy can be administered directly into or in the immediate vicinity of a tumor. Agents of the invention are typically administered to a patient or subject either intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, orally, topically, locally, by inhalation, by injection, by infusion, by continuous infusion, by localized perfusion bathing target cells directly, via a catheter, via a lavage or combinations thereof Embodiments of the invention contemplate administration of the cell cycle modulating agent, the anti-cancer therapy, or both the cell cycle modulating agent and the anti-cancer therapy more than 1, 2, 3, 4, 5, 6, 7, 8, 9, 10 or more times over 1, 2, 3, 4, 5, 6, 7, 8, 9, 10, 11, 12, 13, 14, 15, 16, 17, 18, 19, 20, 21, 22, 23, 24 or more hours, days, or weeks.

Methods of the invention also include methods for treating a subject at a heightened risk of cancer or identified as having a heightened risk of cancer comprising providing an effective amount of a cell cycle modulator and anti-cancer therapy or agent to the subject, wherein the amount of the cell cycle modulator and anticancer therapy is sufficient to reduce the risk of cancer or the recurrence of cancer in the subject.

Other methods of the invention include methods for treating or reducing cancer metastasis in a subject comprising administering to the subject an effective amount of: a cell cycle modulator (particularly an adenovirus and more particularly an oncolytic adenovirus) capable of being expressed in the subject; and an effective amount of an anti-cancer therapy.

Still other methods of the invention include methods for treating a premalignant lesion in a subject comprising providing an effective amount of a cell cycle modulating agent and an anti-cancer therapy or agent to the subject.

In certain aspects of the invention a subject has, is diagnosed with, is suspected of having, or has a propensity for developing cancer. The cancer can be a astrocytoma, oligodendroglioma, anaplastic glioma, glioblastoma, ependymoma, meningioma, pineal region tumor, choroid plexus tumor, neuroepithelial tumor, embryonal tumor, peripheral neuroblastic tumor, tumor of cranial nerves, tumor of the hemopoietic system, germ cell tumors, tumor of the sellar region or brain metastases from lung, breast, kidney, colon, ovarian cancers, melanoma, and sarcomas. In certain aspects the cancer is a cancer of the nervous system, particularly glioblastoma.

In some embodiments, a subject is given about, less than about, or at most about 0.005, 0.05, 0.1, 0.2, 0.3, 0.4, 0.5, 0.6, 0.7, 0.8, 0.9, 1.0, 2, 3,4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 100, 110, 120, 130, 140, 150 nM/kg/day, or any range derivable therein of an agent, so long as a effect is being mediated. Alternatively, the amount of an cell cycle modulator anti-cancer agent that is administered can be expressed in terms of nanogram (ng). In certain embodiments, the amount given is about, less than about, or at most about 0.005, 0.05, 0.1, 0.2, 0.3, 0.4, 0.5, 0.6, 0.7, 0.8, 0.9, 1.0, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 ng/kg/day, or any range derivable therein, so long as a genomic effect is being mediated.

Adenovirus can be administered in amounts of about 10³ to about 10¹⁵ viral particles, from about 10⁵ to about 10¹², from about 10⁷ to about 10¹⁰ viral particles or ranges there between to subject.

The methods can further comprise determining the proportion of cells in S-phase. In certain aspects at least 40, 45, 50, 55, 60, 65, 70, 75, 80, 85, 90, or 95% of cells in a biopsy sample are in S-phase.

In further embodiments the invention includes composition that comprise at least 1, 2, 3, 4, 5, or more cell cycle modulating agents in combination with 1, 2, 3, 4, 5, 6, 7, 8, 9, 10 or more anticancer agents or agents that sensitize a cancer cell to an anticancer agent. Preferably these composition will be in pharmaceutically acceptable carriers.

Certain S-phase specific antimetabolites and M-phase specific vinca alkaloids are well known as effective antineoplastic agents [See Corr, R. T., and Fritz, W. L., “CANCER CHEMOTHERAPY HANDBOOK”, 1980, Elseveir North Holland, Inc., New York, N.Y. and Calabresi, P., and Chabner, B. A., “CHEMOTHERAPY OF NEOPLASTIC DISEASES”, Section XII, GOODMAN AND GILLMAN'S THE PHARMACOLOGICAL BASIS OF THERAPEUTICS, 8th ed., 1990, Pergamon Press Inc., Elmsford, N.Y.]. Cytarabine (ARA-C), fluorouracil (5-FU), mercaptopurine (6-MP), methotrexate (MTX), thioguanine (6-TG), hydroxyurea, prednisone, procarbazine and diglycoaldehyde are examples of antimetabolites with antineoplastic properties. Vincristine and vinblastine are examples of vinca alkaloids with antineoplastic properties. These agents are proven to be useful in the treatment of patients suffering from a variety of neoplastic disease states.

It will be understood that “an effective amount” means that the subject, including patients, is provided with an amount or amounts of one or more compositions that lead to a therapeutic benefit. It will be understood that the subject may given an amount of cell cycle modulator and an amount of an anti-cancer therapy, both in amounts that contribute to a therapeutic benefit. In embodiments, in which more than two different compounds are provided the term “effective amount” means that subject is provided with an amount that provides a therapeutic benefit as a result of the amount of the combination of substances that is provided to the subject.

“Treatment” and “treating” refer to administration or application of an agent, drug, or remedy to a subject or performance of a procedure or modality on a subject for the purpose of obtaining a therapeutic benefit of a disease or health-related condition.

The term “therapeutic benefit” used throughout this application refers to anything that promotes or enhances the well-being of a subject with respect to the medical treatment of his/her condition, which includes, but is not limited to, treatment of pre-cancer, dysplasia, cancer, and other hyperproliferative diseases. A list of nonexhaustive examples of therapeutic benefit includes extension of the subject's life by any period of time, decrease or delay in the neoplastic development of the disease, decrease in hyperproliferation, reduction in tumor growth, delay of metastases or reduction in number of metastases, reduction in cancer cell or tumor cell proliferation rate, decrease or delay in progression of neoplastic development from a premalignant condition, and a decrease in pain to the subject that can be attributed to the subject's condition.

“Prevention” and “preventing” are used according to their ordinary and plain meaning to mean “acting before” or such an act. In the context of a particular disease or health-related condition, those terms refer to administration or application of an agent, drug, or remedy to a subject or performance of a procedure or modality on a subject for the purpose of blocking the onset of a disease or health-related condition. An amount of a pharmaceutical composition that is suitable to prevent a disease or condition is an amount that is known or suspected of blocking the onset of the disease or health-related condition.

A subject or patient can be a subject or patient who is known or suspected of being free of a particular disease or health-related condition at the time the relevant preventive agent is administered. The subject, for example, can be a subject with no known disease or health-related condition (i.e., a healthy subject). In some embodiments, the subject is a subject at risk of developing a particular disease or health-related condition. For example, the subject may have a history of cancer that has been treated in the past and is at risk of developing a recurrence of the cancer. The subject may be a subject at risk of developing a recurrent cancer because of a genetic predisposition or as a result of past chemotherapy. Alternatively, the subject may be a subject with a history of successfully treated cancer who is currently disease-free, but who is at risk of developing a second primary tumor. For example, the risk may be the result of past radiation therapy or chemotherapy that was applied as treatment of a first primary tumor. In some embodiments, the subject may be a subject with a first disease or health-related condition, who is at risk of development of a second disease or health-related condition.

“Synergistic” indicates that the therapeutic effect is greater than would have been expected based on adding the effects of each agent applied as a monotherapy.

The term “subject” includes any human, patient, or animal with, having, or is suspected of having or developing a disease or health related condition. In particular, a patient is a subject that has cancer is or will undergo treatment. In many embodiments of the invention, a subject is a mammal, specifically a human.

The term “provide” is used according to its ordinary and plain meaning: “to supply or furnish for use” (Oxford English Dictionary). The term “purified” or “isolated” means that component was previously isolated away or purified from other proteins and that the component is at least about 95% pure prior to being formulated in the composition. In certain embodiments, the purified or isolated component is about or is at least about 95, 96, 97, 98, 99, 99.1, 99.2, 99.3, 99.4, 99.5% pure or more, or any range derivable therein.

Embodiments discussed in the context of a methods and/or composition of the invention may be employed with respect to any other method or composition described herein. Thus, an embodiment pertaining to one method may be applied to other methods of the invention as well.

The term “about” refers to the imprecision of determining virus, protein or other amounts and measures, and is intended to include at least one standard deviation of error for any particular assay, measure or quantification.

“A” or “an,” as used herein in the specification, may mean one or more than one. As used herein in the claim(s), when used in conjunction with the word “comprising,” the words “a” or “an” may mean one or more than one.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein:

FIG. 1 illustrates an exemplary survival curve related to Delta-24 alone or in combination with temodar.

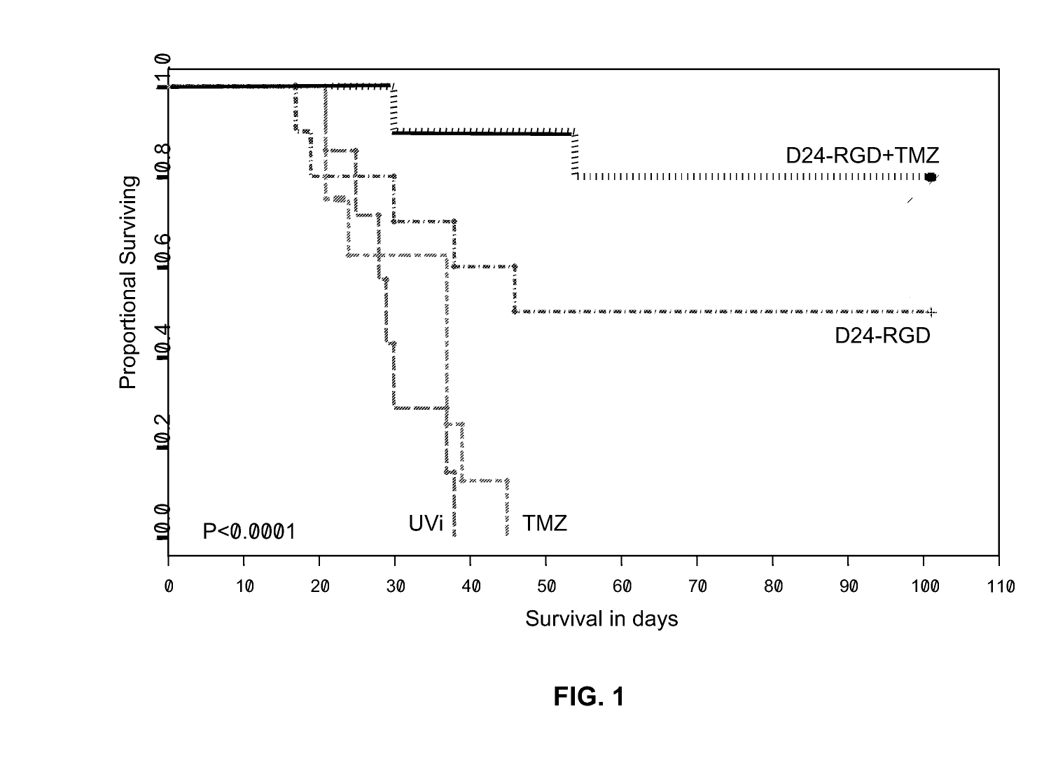

FIG. 2 illustrates an exemplary survival curve related to ICOVIR-5 alone or in combination with tmz.

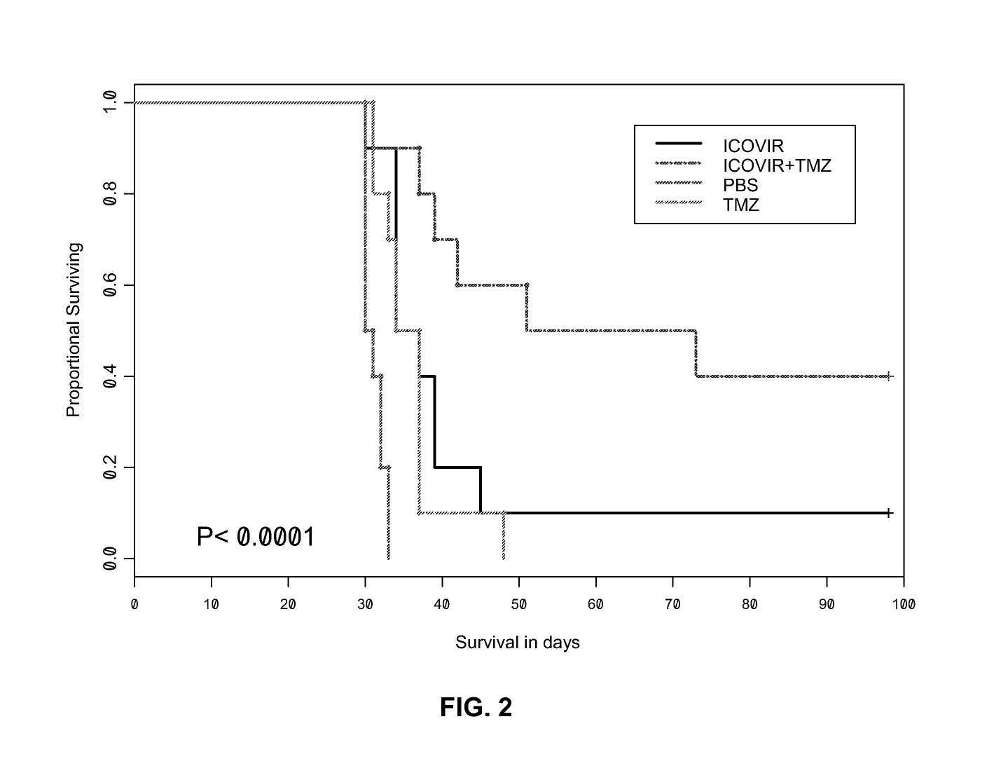

### DETAILED DESCRIPTION

Malignant tumors that are intrinsically resistant to conventional therapies are significant therapeutic challenges. Such malignant tumors include, but are not limited to malignant gliomas and recurrent systemic solid tumors such as lung cancer. Malignant gliomas are the most abundant primary brain tumors having an annual incidence of 6.4 cases per 100,000 (CBTRUS, 2002-2003). These neurologically devastating tumors are the most common subtype of primary brain tumors and are one of the deadliest human cancers. In the most aggressive cancer, manifestation glioblastoma multiforme (GBM), median survival duration for patients ranges from 9 to 12 months, despite maximum treatment efforts (Hess et al., 1999). A prototypic disease, malignant glioma is inherently resistant to current treatment regimens (Shapiro and Shapiro, 1998). In fact, in approximately ⅓ of patients with GBM the tumor will continue to grow despite treatment with radiation and chemotherapy. Median survival even with aggressive treatment including surgery, radiation, and chemotherapy is less than 1 year (Schiffer, 1998). Because few good treatment options are available for many of these refractory tumors, the exploration of novel and innovative therapeutic approaches is essential.

One potential method to improve treatment is based on the concept that naturally occurring viruses can be engineered to produce an oncolytic effect in tumor cells (Wildner, 2001; Jacotat, 1967; Kim, 2001; Geoerger et al., 2002; Yan et al., 2003; Vile et al., 2002, each of which is incorporated herein by reference). In the case of adenoviruses, specific deletions within their adenoviral genome can attenuate their ability to replicate within normal quiescent cells, while they retain the ability to replicate in tumor cells. One such conditionally replicating adenovirus, A24, has been described by Fueyo et al. (2000), see also U.S. Patent Application No. 20030138405, each of which are incorporated herein by reference. The A24 adenovirus is derived from adenovirus type 5 (Ad-5) and contains a 24-base-pair deletion within the CR2 portion of the E1A gene. Significant antitumor effects of Δ24 have been shown in cell culture systems and in malignant glioma xenograft models.

Oncolytic adenoviruses include conditionally replicating adenoviruses (CRADs), such as Delta 24, which have several properties that make them candidates for use as biotherapeutic agents. One such property is the ability to replicate in a permissive cell or tissue, which amplifies the original input dose of the oncolytic virus and helps the agent spread to adjacent tumor cells providing a direct antitumor effect.

Embodiments of the present invention couple the oncolytic component of Delta 24 with a transgene expression approach to produce an armed Delta 24. Armed Delta 24 adenoviruses may be used for producing or enhancing bystander effects within a tumor and/or producing or enhancing detection/imaging of an oncolytic adenovirus in a patient, or tumor associated tissue and/or cell. It is contemplated that the combination of oncolytic adenovirus with various transgene strategies will improve the therapeutic potential against a variety of refractory tumors, as well as provide for improved imaging capabilities. In certain embodiments, an oncolytic adenovirus may be administered with a replication defective adenovirus, another oncolytic virus, a replication competent adenovirus, and/or a wildtype adenovirus. Each of which may be administered concurrently, before or after the other adenoviruses.

Embodiments of the invention include the Delta 24 adenovirus comprising an expression cassette containing a heterologous gene. Examples of such heterologous genes include therapeutic genes, pro-drug converting enzymes, cytosine deaminase (to convert 5-FC to 5-FU), a yeast cytosine deaminase, a humanized yeast cytosine deaminase, an image enhancing polypeptides, a sodium-iodide symporter, anti-sense or ihibitory VEGF, Bcl-2, Ang-2, or interferons alpha, beta or gamma. In certain aspects of the present invention, a Delta 24 oncolytic adenoviral strategy is coupled with an Ang-2 transgene, sodium-iodide symporter (NIS) transgene, humanized yeast CD or a yeast CD transgene approach for augmenting bystander effects and/or obtaining imaging of the replicating virus within an in vivo tumor setting.

Tumor-selective replication is one of the most relevant advances in adenovirus-based anticancer therapies. The oncolytic virus is itself capable of lysing the infected tumor cell to eradicate or reduce tumor mass. Replication amplifies the input dose of the oncolytic virus and helps disseminate the agent to adjacent tumor cells. The inventors have described previously the oncolytic adenovirus, Delta-24, which expresses a mutant E1A protein that is unable to bind to Rb (see U.S. patent applications Ser. 10/124,608, filed Apr. 17, 2002 and Ser. No. 11/080,248, filed Mar. 15, 2005). Because of its inability to bind to Rb, Delta-24 behaves like a wild-type adenovirus in cancer cells but does not replicate efficiently in nondividing normal cells. It has been reported that adenoviruses infect primarily quiescent cells and then induce them to enter the S phase of the cell cycle so that viral DNA synthesis can occur (Flint and Shenk, 1997; Gomez-Manzano et al., 2004). This ability to induce quiescent cells to enter the S phase makes these viruses attractive for use with agents, such as topoisomerase I inhibitors, which target cells in the S phase. Of particular interest, Delta-24 induces the accumulation of infected cancer cells in the S phase (Fueyo et al.,2000). Previous studies have shown that the level of topoisomerase I expression correlates with sensitivity to the topoisomerase inhibitor camptothecin in some tumor cells (Sugimoto et al., 1990). Topoisomerase I inhibitors are a class of agents that interfere with DNA “unwinding” during DNA replication and RNA transcription and stabilize DNA-topoisomerase I complexes through noncovalent interactions to yield enzyme-linked DNA singlestrand breaks. The prolonged exposure of replicating cells to these agents produces lethal dsDNA breaks that can trigger programmed cell death (D'Arpa et al., 1990). Therefore, strategies that upregulate topoisomerase I protein levels and activity could enhance the effects of topoisomerase I-dependent chemotherapy. It has been reported that adenovirus infection also elevates cellular topoisomerase I levels, making adenoviruses even more attractive in combination with S-phase-specific agents (Romig and Richter, 1990; Chow and Pearson, 1985). In this study, we sought to determine in vitro and in vivo whether Delta-24 could both sensitize glioma cells to the camptothecin analogue irinotecan (CPT-11) by up-regulating topoisomerase I expression and inducing cancer cells to accumulate in the S phase. Our results showed that the infection of cancer cells with Delta-24 resulted in the marked accumulation of cells in the S phase and in an increase in the levels and activity of topoisomerase I in human glioma cells. Further, the inventors found that the sequential administration of Delta-24 and CPT-11 significantly prolonged the survival of gliomabearing animals. The study therefore showed that there is a rational basis for the combination of adenoviral therapy and chemotherapy and that the anticancer effect of the two agents was enhanced when given in combination.

Delta-24 infection enhanced expression and activity of topoisomerase I. The inventors investigate whether Delta-24 adenovirus could sensitize glioma cells to the camptothecin analogue CPT-11 by up-regulation of topoisomerase I expression. The expression of topoisomerase I was assessed in the U-87 MG and U-251 MG human glioma cells after infection with Delta-24. These two cell lines were selected because they were used previously to characterize the antiglioma effect of Delta-24 (Fueyo et al., 2000). Western blot analysis showed that endogenous topoisomerase I was expressed at a low level in both glioma cell lines. However, treatment with Delta-24 resulted in at least a 4-fold increase in topoisomerase levels in both cell lines compared with mock-infected or UVi Delta-24-infected cells. Cells infected with the wild-type adenovirus exhibited an increase in topoisomerase I expression similar to that seen in the Delta-24- infected cells.

The inventors determined whether Delta-24 infection resulted in increased topoisomerase I activity in glioma cells in culture. A plasmid DNA used as a template for the topoisomerase I reaction incubated with UVi Delta-24-infected nuclear extracts appeared predominantly in the supercoiled form, similar to the finding in the control cells containing the form I DNA plasmid without topoisomerase I. In addition, Delta-24-infected nuclear extracts from both glioma cultures displayed a topoisomerase I activity that caused the plasmidic DNA to relax comparable with the finding in the topoisomerase I-treated positive controls. Taken together, these observations indicate that infection with the Delta-24 adenovirus increases topoisomerase I protein levels and activity.

Cell cycle profile of Delta-24- and CPT-11-treated cells. Previous data showed that Delta-24 infections cause cells to accumulate in the S phase of the cell cycle (Fueyo et al., 2000). In this study, U-87 MG and U-251 MG human glioma cells were infected with Delta-24 adenovirus and treated 2 days later with CPT-1 1. Cells were then collected and their DNA content was examined by flow cytometry. As expected, the accumulation of Delta-24-infected cells in the S phase of the cell cycle was striking (>70% of the cells in culture) and statistically significant in comparison with the control cells infected with UVi Delta-24 (P<0.001; Table 1). It was also shown that treatment with CPT-11 resulted in an accumulation of cells in the G2-M phase (>55% of the cells in culture; P<0.001, compared with vehicle-treated cells). The inventors investigated the effect of the combination of the Delta-24 adenovirus and CPT-11 on cell cycle progression. Cells infected with Delta-24 and then treated 48 hours later with CPT-11 showed an overrepresentation of cells in the S phase (>65% of the cells in culture) with a dramatic decrease in the G2-M population (<20% of the cells in culture). Thus, cells treated with a combined regimen exhibited a cell cycle profile similar to that of cells treated with Delta-24 alone.

These data indicate that Delta-24 infection overrides the G2-M arrest induced by CPT-11 and maintains a large population of cells in the S phase, suggesting that Delta-24-infected cells are likely to be particularly susceptible to S-phase-based chemotherapeutic agents.

Effect of CPT-11 and Delta-24 on proliferation of human glioma cells. The inventors ascertained the sensitivity of U-87 MG and U-251 MG glioma cells to CPT-11. In both cell lines, CPT-11 inhibited cell proliferation in a concentration dependent fashion as assessed by the 3-(4,5-dimethylthiazol-2-yl)-2,5-diphenyltetrazolium bromide assay. In contrast, no significant inhibition was seen in control cells treated with equivalent concentrations of vehicle in the absence of CPT-11 (data not shown). After this, the effect of the sequential administration of Delta-24 combined with several different CPT-11 concentrations was assessed. For these experiments, the inventors designed a treatment schedule based on the hypothetical mechanism of the Delta-24-mediated potentiation of the drug effect (i.e., induction of topoisomerase I) and previous data indicating that the greatest accumulation of cells in the S phase occurs within 48 hours of Delta-24 infection (Fueyo et al., 2000). Thus, cells were infected with Delta-24 at a range of 1 to 10 MOIs, and CPT-11 was added 48 hours later. The IC₅₀ dose of CPT-11 decreased from 3.4 μmol/L in U-87 MG cells infected with UVi Delta-24 to 1.5 μmol/L in Delta-24-infected cells infected at a dose of 10 MOIs (P<0.001) and from 7.2 μmol/L in UVi Delta-24-infected U-251 MG cells to 1 Amol/L in Delta-24-infected cells infected with 10 MOIs (P<0.001). The IC₅₀ for CPT-11 was modified significantly (to ˜2.5 μmol/L) in both U-87 MG and U-251 MG cells infected with 2 MOIs of Delta-24.

Importantly, in an independent set of experiments, Delta-24 was tested as a potentiator of the CPT-11-mediated cytotoxicity in glioma cultures. In this experiment in which low doses of both Delta-24 (2 MOIs) and CPT-11 (2.5 μmol/L) were used, it was observed that the effect of the combination of the two agents exceeded the total effect of the two when given alone in both U-87 MG and U-251 MG cells. Sequential administration of Delta-24 and CPT-11 did not modify the replication capability of the adenovirus. Viral replication assays done after the combined treatment of glioma cells with Delta-24 and CPT-11 showed that there was no significant modification in the replicative phenotype of the oncolytic adenovirus when it was combined with the topoisomerase I inhibitor under the conditions used in this experiment. Specifically, the resulting viral titers after both treatments differed by only 0.13±0.37 and 0.63±0.45 orders of magnitude in the U-87 MG and U-251 MG cells, respectively. These results show that Delta-24 replicated with a similar efficiency whether given singly or in combination with CPT-11 (P>0.1; double-sided t test). This indicated that the anticancer effect observed in cells treated with Delta-24 and CPT-11 could be the result of the Delta-24-mediated potentiation of the effect of CPT-11.

Combined antiglioma effect of Delta-24 and CPT-11 in vivo. To assess the potential therapeutic relevance of the findings from the in vitro studies, the Delta-24/CPT-11 sequential treatment was tested in an in vivo model of a human glioma xenograft implanted intracranially that had been validated for testing the antiglioma effect of Delta-24 (Fueyo et al., 2003). On day 3 after U-87 MG cell implantation, animals were treated with a single intratumoral injection of Delta-24 or UVi Delta-24 (1.5×10⁸ viral particles in 5 μL). CPT-11 was given on days 7, 12, and 20 after cell implantation (5 mg/kg i.p.). The median survival was 27 days in the control group of animals (treated with vehicle plus UVi Delta-24), and all these animals died by day 32. Treatment with CPT-11 (plus UVi Delta-24) or a single dose of Delta-24 (plus vehicle) extended the survival by an average of 4 and 8 days, respectively (P=0.001 and P<0.0001, respectively, compared with vehicle-treated animals). The combination treatment consisting of Delta-24 followed by CPT-11 resulted in the most substantial increase in animal survival (median overall survival of 42 days). In addition, the overall survival of the animals treated with the combined therapy differed significantly from that in animals treated with either agent alone (P<0.005, log-rank test), as did differences in the 60-day survival rate (P<0.012, Fisher's test). Of further relevance, there were no long-term survivors among the animals receiving a single-treatment regimen; however, 7 of 31 (22.5%) animals treated with the combination of Delta-24 and CPT-11 survived >3 months after tumor implantation without showing any sign of neurologic distress. Examination of the brains of the animals showed that all animals that died had evidence of intracranial tumor. In contrast, microscopic examination of the brains of the longterm surviving animals that received the combination treatment showed complete tumor regression. In these animals, however, sequelae of the tumors were identified, including dystrophic calcification and microcyst formation, at the tumor implantation site in the right caudate nucleus. Immunohistochemical analyses of the brains of the long-term survivors using both anti-E1A and anti-hexon antibodies failed to detect viral particles (data not shown). Further, E1A expression or signs of inflammation in the normal brain tissue was not observed.

Oncolytic adenoviruses are alternative promising therapies for the treatment of gliomas. Nevertheless, the effective treatment of gliomas with oncolytic adenovirus has been hampered by the relative low persistence of the vectors, difficulty in systemic delivery and side toxicity due to undesired targeting of normal cells and to the immune system response. One strategy to improve the efficacy of oncolytic adenoviruses is to combine them with chemotherapeutic drugs. The inventors have generated an adenovirus, termed ICOVIR, that encompasses three elements: enhanced tropism trough integrin infection (RGD-4C modification of the fiber HI loop), tumor selectivity (Delta-24 mutation in the Rb-binding CR2 region of E1A), and decrease of E1A-mediated toxicity (insertion of two E2F1 promoter sequences preceded by an insulator upstream of the E1A coding sequence in substitution of the native E1A promoter) to provide the construct with both cell-specific gene expression and strong viral replication capability in cancer cells. In this regard, ICOVIR-5 probed to be a highly selective vector in gliomas at the same time that retained a robust cell killing potential and a negligible toxicity in vitro and which is more important in vivo. To test the hypothesis that ICOVIR-5 infection favors the effect of temozolomide (TMZ) and RAD001, we first examine in vitro the effect of ICOVIR-5 alone or in combination with TMZ or RAD001 in U87-MG and U251-MG glioma cell lines by MTT. Our data showed that both drugs were more efficient in combination with ICOVIR-5 that when administer alone. In addition, ICOVIR-5 replication properties were not affected by the addition either drug. Of interest, individually systemic administration of ICOVIR-5 resulted in improved survival and generation of long-term survivors (animal living more than 100 days after treatment). Importantly, the combination of the drugs with ICOVIR-5 resulted in a significant increased in the median survival (P<0.001) and in 40% of long time survivors. Examination of the brains by IHC showed E1A and hexon staining meaning the ability of the virus to infect and efficiently replicate in combination with the drugs. Of importance, all the regimens showed very low hepatotoxicity as probed by the low expression levels of E1A in liver and serum levels of AST and ALT similar to the physiological values. The inventors believe that the combination of ICOVIR and chemotherapy allows for multi-compartmental treatment. The antiangiogenic, cytostatic and immunosuppressant effect of the drugs facilitates the local spread of the virus within the tumour and into the surrounding brain areas that may contain invading cells from the glioma at the same time that generate a wider window of time for the virus to elicit an oncolytic effect. In summary, ICOVIR-5 in combination with TMZ and RAD001 constitutes a promising strategy for the treatment of gliomas.

ICOVIR-5

ICOVIR-5 is a new oncolytic adenovirus that encompasses three elements: enhanced tropism through integrin infection (RGD-4C modification of the fiber HI loop), tumor selectivity (Delta-24 mutation in the Rb-binding CR2 region of E1A), and decrease of E1A-mediated toxicity (insertion of two E2F1 promoter sequences preceded by an insulator upstream of the E1A coding sequence). ICOVIR-5 probed to be a highly selective vector in gliomas at the same time that retained a robust cell killing potential and a negligible toxicity in vitro and in vivo. Of interest, treatment of glioma xenografts with ICOVIR-5 resulted in improved survival and generation of long-term survivors. Importantly, the combination of TMZ with ICOVIR-5 resulted in a significant increased in the median survival (P<0.001) and in 40% of long time survivors.

### Therapeutic Agents

Anticancer Agents

Examples of anti-angiogenesis agents include, but are not limited to, retinoid acid and derivatives thereof, 2-methoxyestradiol, ANGIOSTATIN^{R} protein, ENDOSTATIN^{R} protein, suramin, squalamine, tissue inhibitor of metalloproteinase-I, tissue inhibitor of metalloproteinase-2, plasminogen activator inhibitor-1, plasminogen activator inhibitor-2, cartilage-derived inhibitor, paclitaxel, platelet factor 4, protamine sulphate (clupeine), sulphated chitin derivatives (prepared from queen crab shells), sulphated polysaccharide peptidoglycan complex (sp-pg), staurosporine, modulators of matrix metabolism, including for example, proline analogs ((1-azetidine-2-carboxylic acid (LACA), cishydroxyproline, d,1-3,4-dehydroproline, thiaproline], α,α-dipyridyl, β-aminopropionitrile fumarate, 4-propyl-5-(4-pyridinyl)-2(3h)-oxazolone; methotrexate, mitoxantrone, heparin, interferons, 2 macroglobulin-serum, chimp-3, chymostatin, beta.- cyclodextrin tetradecasulfate, eponemycin; fumagillin, gold sodium thiomalate, d-penicillamine (CDPT), β-1-anticollagenase-serum, α-2-antiplasmin, bisantrene, lobenzarit disodium, n-(2-carboxyphenyl-4- chloroanthronilic acid disodium or “CCA”, thalidomide; angostatic steroid, cargboxynaminolmidazole; metalloproteinase inhibitors such as BB94. Other anti-angiogenesis agents include antibodies, preferably monoclonal antibodies against these angiogenic growth factors: bFGF, aFGF, FGF-5, VEGF isoforms, VEGF-C, HGF/SF and Ang-1/Ang-2. (Ferrara and Alitalo (1999) Nature Medicine 5:1359-1364. Calbiochem (San Diego, Calif.) carries a variety of angiogensis inhibitors including (catalog number/product name) 658553/AG 1433; 129876/Amiloride, Hydrochloride; 164602/Aminopeptidase N Inhibitor; 175580/Angiogenesis Inhibitor; 175602/Angiogenin (108-123); 175610/Angiogenin Inhibitor; 176600/Angiopoietin-2, His•Tag®, Human, Recombinant, Mouse, Biotin Conjugate; 176705/Angiostatin K1-3, Human; 176706/Angiostatin K1-5, Human; 176700/Angiostatin® Protein, Human; 178278/Apigenin; 189400/Aurintricarboxylic Acid; 199500/Benzopurpurin B; 211875/Captopril; 218775/Castanospermine, Castanospermum australe; 251400/D609, Potassium Salt; 251600 Daidzein; 288500/DL-a-Difluoromethylornithine, Hydrochloride; 324743/Endostatin™ Protein, His•Tag®, Mouse, Recombinant, Spodoptera frugiperda; 324746/Endostatin™ Protein, Human, Recombinant, Pichia pastoris; 324733/Endostatin™ Protein, Mouse, Recombinant, Pichia pastoris; 329740 Eriochrome® Black T Reagent; 344845 Fumagillin, Aspergillus fumigatus; 345834 Genistein; 375670/Herbimycin A, Streptomyces sp.; 390900/4-Hydroxyphenylretinamide; 407293/a-Interferon, Mouse, Recombinant, E. coli; 407306/g-Interferon, Human, Recombinant, E. coli; 05-23-3700/Laminin Pentapeptide; 05-23-3701/Laminin Pentapeptide Amide; 428150/Lavendustin A; 454180/2-Methoxyestradiol; 475838/Mifepristone; 475843/Minocycline, Hydrochloride; 4801/Neomycin Sulfate; 521726/Platelet Factor 4, Human Platelets; 553400/Radicicol, Diheterospora chlamydosporia; 554994/RHC-80267; 565850/Shikonin; 573117/SMC Proliferation Inhibitor-2w; 572888/SU1498; 572632/SU5614; 574625/Suramin, Sodium Salt; 608050/TAS-301; 585970/(±)-Thalidomide; 605225/Thrombospondin, Human Platelets; 616400/Tranilast; 654100/TSR1265; 676496/VEGF Inhibitor, CBO-P11; 676493/VEGF Inhibitor, Flt2-11; 676494 VEGF Inhibitor, Je-11; 676495 VEGF Inhibitor, VI; 676480/VEGF Receptor 2 Kinase Inhibitor I; 676485/VEGF Receptor 2 Kinase Inhibitor II; 676475/VEGF Receptor Tyrosine Kinase Inhibitor, and other such agents known to those of ordinary skill in the medical arts.

Therapeutic Genes

Aspects of the invention include nucleic acids or genes that encode a detectable and/or therapeutic polypeptide for use in anti-cancer gene therapy. In certain embodiments of the present invention, the gene is a therapeutic, or therapeutic gene. A “therapeutic gene” is a gene which can be administered to a subject for the purpose of treating or preventing a disease. For example, a therapeutic gene can be a gene administered to a subject for treatment or prevention of diabetes or cancer. Examples of therapeutic genes include, but are not limited to, Rb, CFTR, p16, p21, p27, p57, p73, C-CAM, APC, CTS-1, zac1, scFV ras, DCC, NF-1, NF-2, WT-1, MEN-I, MEN-II, BRCA1, VHL, MMAC1, FCC, MCC, BRCA2, IL-1, IL-2, IL-3, IL-4, IL-5, IL-6, IL-7, IL-8, IL-9, IL-10, IL-11 IL-12, GM-CSF, G-CSF, thymidine kinase, mda7, fus, interferon α, interferon β, interferon γ, ADP, p53, ABLI, BLC1, BLC6, CBFA1, CBL, CSFIR, ERBA, ERBB, EBRB2, ETS1, ETS2, ETV6, FGR, FOX, FYN, HCR, HRAS, JUN, KRAS, LCK, LYN, MDM2, MLL, MYB, MYC, MYCL1, MYCN, NRAS, PIM1, PML, RET, SRC, TAL1, TCL3, YES, MADH4, RB1, TP53, WT1, TNF, BDNF, CNTF, NGF, IGF, GMF, aFGF, bFGF, NT3, NT5, ApoAI, ApoAIV, ApoE, Rap1A, cytosine deaminase, Fab, ScFv, BRCA2, zac1, ATM, HIC-1, DPC-4, FHIT, PTEN, ING1, NOEY1, NOEY2, OVCA1, MADR2, 53BP2, IRF-1, Rb, zac1, DBCCR-1, rks-3, COX-1, TFPI, PGS, Dp, E2F, ras, myc, neu, raf, erb, fms, trk, ret, gsp, hst, abl, E1A, p300, VEGF, FGF, thrombospondin, BAI-1, GDAIF, or MCC.

In certain embodiments of the present invention, the therapeutic gene is a tumor suppressor gene. A tumor suppressor gene is a gene that, when present in a cell, reduces the tumorigenicity, malignancy, or hyperproliferative phenotype of the cell. This definition includes both the full length nucleic acid sequence of the tumor suppressor gene, as well as non-full length sequences of any length derived from the full length sequences. It being further understood that the sequence includes the degenerate codons of the native sequence or sequences which may be introduced to provide codon preference in a specific host cell.

Examples of tumor suppressor nucleic acids within this definition include, but are not limited to APC, CYLD, HIN-1, KRAS2b, p16, p19, p21, p27, p27mt, p53, p57, p73, PTEN, Rb, Uteroglobin, Skp2, BRCA-1, BRCA-2, CHK2, CDKN2A, DCC, DPC4, MADR2/JV18, MEN1, MEN2, MTS1, NF1, NF2, VHL, WRN, WT1, CFTR, C-CAM, CTS-1, zac1, scFV, MMAC1, FCC, MCC, Gene 26 (CACNA2D2), PL6, Beta* (BLU), Luca-1 (HYAL1), Luca-2 (HYAL2), 123F2 (RASSF1), 101F6, Gene 21 (NPRL2), or a gene encoding a SEM A3 polypeptide and FUS1. Other exemplary tumor suppressor genes are described in a database of tumor suppressor genes at www.cise.ufl.edu/˜yy1/HTML-TSGDB/Homepage.html. This database is herein specifically incorporated by reference into this and all other sections of the present application. Nucleic acids encoding tumor suppressor genes, as discussed above, include tumor suppressor genes, or nucleic acids derived therefrom (e.g., cDNAs, cRNAs, mRNAs, and subsequences thereof encoding active fragments of the respective tumor suppressor amino acid sequences), as well as vectors comprising these sequences. One of ordinary skill in the art would be familiar with tumor suppressor genes that can be applied in the present invention.

In certain embodiments of the present invention, the therapeutic gene is a gene that induces apoptosis (i.e., a pro-apoptotic gene). A “pro-apoptotic gene amino acid sequence” refers to a polypeptide that, when present in a cell, induces or promotes apoptosis. The present invention contemplates inclusion of any pro-apoptotic gene known to those of ordinary skill in the art. Exemplary pro-apoptotic genes include CD95, caspase-3, Bax, Bag-1, CRADD, TSSC3, bax, hid, Bak, MKP-7, PERP, bad, bcl-2, MST1, bbc3, Sax, BIK, BID, and mda7. One of ordinary skill in the art would be familiar with pro-apoptotic genes, and other such genes not specifically set forth herein that can be applied in the methods and compositions of the present invention.

The therapeutic gene can also be a gene encoding a cytokine. The term ‘cytokine’ is a generic term for proteins released by one cell population which act on another cell as intercellular mediators. A “cytokine” refers to a polypeptide that, when present in a cell, maintains some or all of the function of a cytokine. This definition includes full-length as well as non-full length sequences of any length derived from the full length sequences. It being further understood, as discussed above, that the sequence includes the degenerate codons of the native sequence or sequences which may be introduced to provide codon preference in a specific host cell.

Examples of such cytokines are lymphokines, monokines, growth factors and traditional polypeptide hormones. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; prostaglandin, fibroblast growth factor; prolactin; placental lactogen, OB protein; tumor necrosis factor-α and -β; mullerian-inhibiting substance; mouse gonadotropin-associated peptide; inhibin; activin; vascular endothelial growth factor; integrin; thrombopoietin (TPO); nerve growth factors such as NGF-β; platelet-growth factor; transforming growth factors (TGFs) such as TGF-α and TGF-β; insulin-like growth factor-I and -II; erythropoietin (EPO); osteoinductive factors; interferons such as interferon-α, -β, and -γ; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1α, IL-2, IL-3, IL-4, IL-5, IL-6, IL-7, IL-8, IL-9, IL-10 IL-11, IL-12; IL-13, IL-14, IL-15, IL-16, IL-17, IL-18, IL-19, IL-20, IL-24 LIF, G-CSF, GM-CSF, M-CSF, EPO, kit-ligand or FLT-3.

Other examples of therapeutic genes include genes encoding enzymes. Examples include, but are not limited to, ACP desaturase, an ACP hydroxylase, an ADP-glucose pyrophorylase, an ATPase, an alcohol dehydrogenase, an amylase, an amyloglucosidase, a catalase, a cellulase, a cyclooxygenase, a decarboxylase, a dextrinase, an esterase, a DNA polymerase, an RNA polymerase, a hyaluron synthase, a galactosidase, a glucanase, a glucose oxidase, a GTPase, a helicase, a hemicellulase, a hyaluronidase, an integrase, an invertase, an isomerase, a kinase, a lactase, a lipase, a lipoxygenase, a lyase, a lysozyme, a pectinesterase, a peroxidase, a phosphatase, a phospholipase, a phosphorylase, a polygalacturonase, a proteinase, a peptidease, a pullanase, a recombinase, a reverse transcriptase, a topoisomerase, a xylanase, a reporter gene, an interleukin, or a cytokine.

Further examples of therapeutic genes include the gene encoding carbamoyl synthetase I, ornithine transcarbamylase, arginosuccinate synthetase, arginosuccinate lyase, arginase, fumarylacetoacetate hydrolase, phenylalanine hydroxylase, alpha-1 antitrypsin, glucose-6-phosphatase, low-density-lipoprotein receptor, porphobilinogen deaminase, factor VIII, factor IX, cystathione beta.-synthase, branched chain ketoacid decarboxylase, albumin, isovaleryl-CoA dehydrogenase, propionyl CoA carboxylase, methyl malonyl CoA mutase, glutaryl CoA dehydrogenase, insulin, beta.-glucosidase, pyruvate carboxylase, hepatic phosphorylase, phosphorylase kinase, glycine decarboxylase, H-protein, T-protein, Menkes disease copper-transporting ATPase, Wilson's disease copper-transporting ATPase, cytosine deaminase, hypoxanthine-guanine phosphoribosyltransferase, galactose-1-phosphate uridyltransferase, phenylalanine hydroxylase, glucocerbrosidase, sphingomyelinase, α-L-iduronidase, glucose-6-phosphate dehydrogenase, HSV thymidine kinase, or human thymidine kinase.

Therapeutic genes also include genes encoding hormones. Examples include, but are not limited to, genes encoding growth hormone, prolactin, placental lactogen, luteinizing hormone, follicle-stimulating hormone, chorionic gonadotropin, thyroid-stimulating hormone, leptin, adrenocorticotropin, angiotensin I, angiotensin II, β-endorphin, β-melanocyte stimulating hormone, cholecystokinin, endothelin I, galanin, gastric inhibitory peptide, glucagon, insulin, lipotropins, neurophysins, somatostatin, calcitonin, calcitonin gene related peptide, β-calcitonin gene related peptide, hypercalcemia of malignancy factor, parathyroid hormone-related protein, parathyroid hormone-related protein, glucagon-like peptide, pancreastatin, pancreatic peptide, peptide YY, PHM, secretin, vasoactive intestinal peptide, oxytocin, vasopressin, vasotocin, enkephalinamide, metorphinamide, alpha melanocyte stimulating hormone, atrial natriuretic factor, amylin, amyloid P component, corticotropin releasing hormone, growth hormone releasing factor, luteinizing hormone-releasing hormone, neuropeptide Y, substance K, substance P, or thyrotropin releasing hormone.

As will be understood by those in the art, the term “therapeutic gene” includes genomic sequences, cDNA sequences, and smaller engineered gene segments that express, or may be adapted to express, proteins, polypeptides, domains, peptides, fusion proteins, and mutants. The nucleic acid molecule encoding a therapeutic gene may comprise a contiguous nucleic acid sequence of about 5 to about 12000 or more nucleotides, nucleosides, or base pairs.

Modifications of Oncolytic Adenovirus

Modifications of oncolytic adenovirus described herein may be made to improve the ability of the oncolytic adenovirus to treat cancer. The present invention also includes any modification of oncolytic adenovirus that improves the ability of the adenovirus to treat neoplastic cells. Included are modifications to oncolytic adenovirus genome in order to enhance the ability of the adenovirus to infect and replicate in cancer cells by altering the receptor binding molecules.

The absence or the presence of low levels of the coxsackievirus and adenovirus receptor (CAR) on several tumor types can limit the efficacy of the oncolytic adenovirus. Various peptide motifs may be added to the fiber knob, for instance an RGD motif (RGD sequences mimic the normal ligands of cell surface integrins), Tat motif, poly-lysine motif, NGR motif, CTT motif, CNGRL motif, CPRECES motif or a strept-tag motif (Rouslahti and Rajotte, 2000). A motif can be inserted into the HI loop of the adenovirus fiber protein. Modifying the capsid allows CAR-independent target cell infection. This allows higher replication, more efficient infection, and increased lysis of tumor cells (Suzuki et al., 2001, incorporated herein by reference). Peptide sequences that bind specific human glioma receptors such as EGFR or uPR may also be added. Specific receptors found exclusively or preferentially on the surface of cancer cells may used as a target for adenoviral binding and infection, such as EGFRvIII.

Cell surface receptors are attractive candidates for the targeted therapy of cancer. Growth factors and their receptors play important roles in the regulation of cell division, development, and differentiation. Among those receptors, EGFR was the first to be identified as amplified and/or rearranged in malignant gliomas. EGFR gene amplification in gliomas is often accompanied by gene rearrangement, resulting in deletions of the coding region. The most common variant, de2-7 EGFR or EGFRvIII, is characterized by an in-frame deletion of 801-bp spanning exons 2-7 of the coding sequence. This truncation removes 267 amino acids from the extracellular domain, producing a unique junctional peptide, and renders EGFR unable to bind any known ligand. EGFRvIII is expressed on the cell surface and contains a new tumor-specific protein sequence in its extracellular domain (Sugawa et al. 1990; Ekstrand et al. 1992). The frequency of the EGFRvIII expression in human gliomas is around 20 to 40% (Frederick et al. 2000). Several strategies have already been tested as means for binding the EGFRvIII receptor using peptides and antibodies. A peptide (PEPHC1) has been synthesized and tested for binding to EGFRvIII and EGFR (Campa et al., 2000, which is incorporated herein by reference in its entirety). In in vitro assays, PEPHC1 bound the recombinant EGFRvIII extracellular domain or full-length EGFRvIII (solubilized from cell membranes) in preference to native EGFR. Monoclonal antibodies have been developed with specific activity against this mutant receptor (Lorimer et al. 1996). These antibodies are internalized into the cell after receptor binding. Therefore, this receptor is a desirable target for adenoviral tropism since the receptor-binding molecules are efficiently internalized and the mutant form offers the opportunity to develop tumor-selective targeting strategies.

Although none of the reported adenovirus strategies use the EGFRvIII receptor for adenoviral anchorage and internalization, several reports have characterized EGFR as a potential target in cancer cells. In these studies, the adenoviruses redirected to EGFR were more efficient (in some cases by more than 100 fold) and more selective than the adenoviruses using untargeted vectors to infect and transduce cancer cells. One of the systems relevant to this proposal uses our Delta-24 system in combination with EGFR targeting. In this study, Curiel's group (Hemminki et al. 2001) constructed an adenovirus expressing a secretory adaptor capable of retargeting the adenovirus to EGFR, resulting in a more than 150-fold increase in gene transfer. A replication-competent dual-virus system secreting the adaptor displayed increased oncolytic potency in vitro and therapeutic gain in vivo.

Lack of expression in normal cells and achievable targeting using peptides and antibodies make the EGFR and EGFRvIII systems very suitable for the development of targeted oncolytic adenoviruses with high therapeutic indices (Kuan et al., 2001).

Methods for Treating hyperproliferative Conditions

The present invention involves the treatment of hyperproliferative condition, such as cancer. It is contemplated that a wide variety of tumors may be treated using the methods and compositions of the invention, including gliomas, sarcomas, lung, ovary, breast, cervix, pancreas, stomach, colon, skin, larynx, bladder, prostate, and/or brain metastases of such cancer(s), as well as pre-cancerous cells, metaplasias, dysplasias, or hyperplasia.

The term “glioma” refers to a tumor originating in the neuroglia of the brain or spinal cord. Gliomas are derived form the glial cell types such as astrocytes and oligodendrocytes, thus gliomas include astrocytomas and oligodendrogliomas, as well as anaplastic gliomas, glioblastomas, and ependymomas. Astrocytomas and ependymomas can occur in all areas of the brain and spinal cord in both children and adults. Oligodendrogliomas typically occur in the cerebral hemispheres of adults. Gliomas account for 75% of brain tumors in pediatrics and 45% of brain tumors in adults. The remaining percentages of brain tumors are meningiomas, ependymomas, pineal region tumors, choroid plexus tumors, neuroepithelial tumors, embryonal tumors, peripheral neuroblastic tumors, tumors of cranial nerves, tumors of the hemopoietic system, germ cell tumors, and tumors of the sellar region.

Various embodiments of the present invention deal with the treatment of disease states comprised of cells that are deficient in the Rb and/or p53 pathway. In particular, the present invention is directed at the treatment of diseases, including but not limited to retinoblastomas, gliomas, sarcomas, tumors of lung, ovary, cervix, pancreas, stomach, colon, skin, larynx, breast, prostate and metastases thereof.

There are various categories of brain tumors. Glioblastoma multiforme is the most common malignant primary brain tumor of adults. More than half of these tumors have abnormalities in genes involved in cell cycle control. Often there is a deletion in the CDKN2A or a loss of expression of the retinoblastoma gene. Other types of brain tumors include astrocytomas, oligodendrogliomas, ependymomas, medulloblastomas, meningiomas and schwannomas.

In many contexts, it is not necessary that the cell be killed or induced to undergo cell death or “apoptosis.” Rather, to accomplish a meaningful treatment, all that is required is that the tumor growth be slowed to some degree. It may be that the cell's growth is completely blocked or that some tumor regression is achieved. Clinical terms such as “remission” and “reduction of tumor” burden also are contemplated given their normal usage.

The term “therapeutic benefit” refers to anything that promotes or enhances the well-being of the subject with respect to the medical treatment of his/her condition, which includes treatment of pre-cancer, cancer, and hyperproliferative diseases. A list of nonexhaustive examples of this includes extension of the subject's life by any period of time, decrease or delay in the neoplastic development of the disease, decrease in hyperproliferation, reduction in tumor growth, delay of metastases, reduction in cancer cell or tumor cell proliferation rate, and a decrease in pain to the subject that can be attributed to the subject's condition.

Adenoviral Therapies

Those of skill in the art are well aware of how to apply adenoviral delivery to in vivo and ex vivo situations. For viral vectors, one generally will prepare a viral vector stock. Depending on the kind of virus and the titer attainable, one will deliver 1 to 100, 10 to 50, 100-1000, or up to 1×10⁴, 1×10⁵, 1×10⁶, 1×10⁷, 1×10⁸, 1×10⁹, 1×10¹⁰, 1×10¹¹, or 1×10¹² particles to the patient in a pharmaceutically acceptable composition as discussed below.

Various routes are contemplated for various tumor types. Where discrete tumor mass, or solid tumor, may be identified, a variety of direct, local and regional approaches may be taken. For example, the tumor may be directly injected with the adenovirus. A tumor bed may be treated prior to, during or after resection and/or other treatment(s). Following resection or other treatment(s), one generally will deliver the adenovirus by a catheter having access to the tumor or the residual tumor site following surgery. One may utilize the tumor vasculature to introduce the vector into the tumor by injecting a supporting vein or artery. A more distal blood supply route also may be utilized.

The method of treating cancer includes treatment of a tumor as well as treatment of the region near or around the tumor. In this application, the term “residual tumor site” indicates an area that is adjacent to a tumor. This area may include body cavities in which the tumor lies, as well as cells and tissue that are next to the tumor.

Formulations and Routes of Administration to Patients

Where clinical applications are contemplated, it will be necessary to prepare pharmaceutical compositions in a form appropriate for the intended application. Generally, this will entail preparing compositions that are essentially free of pyrogens, as well as other impurities that could be harmful to humans or animals.

One will generally desire to employ appropriate salts and buffers to render delivery vectors stable and allow for uptake by target cells. Aqueous compositions of the present invention comprise an effective amount of the vector to cells, dissolved or dispersed in a pharmaceutically acceptable carrier or aqueous medium. Such compositions also are referred to as inocula. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well know in the art. Except insofar as any conventional media or agent is incompatible with the present invention, its use in therapeutic compositions is contemplated. Supplementary active ingredients also can be incorporated into the compositions.

The active compositions of the present invention may include classic pharmaceutical preparations. Administration of these compositions according to the present invention will be via any common route so long as the target tissue is available via that route. The routes of administration will vary, naturally, with the location and nature of the lesion, and include, e.g., intradermal, transdermal, parenteral, intracranial, intravenous, intramuscular, intranasal, subcutaneous, percutaneous, intratracheal, intraperitoneal, intratumoral, perfusion, lavage, direct injection, and oral administration and formulation. Preferred embodiments include intracranial or intravenous administration. Administration may be by injection or infusion, see Kruse et al. (1994), specifically incorporated by reference, for methods of performing intracranial administration. Such compositions would normally be administered as pharmaceutically acceptable compositions.

An effective amount of the therapeutic agent is determined based on the intended goal, for example, elimination of tumor cells. The term “unit dose” refers to physically discrete units suitable for use in a subject, each unit containing a predetermined-quantity of the therapeutic composition calculated to produce the desired responses, discussed above, in association with its administration, i.e., the appropriate route and treatment regimen. The quantity to be administered, both according to number of treatments and unit dose, depends on the subject to be treated, the state of the subject and the protection desired. Precise amounts of the therapeutic composition also depend on the judgment of the practitioner and are peculiar to each individual. The engineered viruses of the present invention may be administered directly into animals, or alternatively, administered to cells that are subsequently administered to animals.

As used herein, the term in vitro administration refers to manipulations performed on cells removed from an animal, including, but not limited to, cells in culture. The term ex vivo administration refers to cells that have been manipulated in vitro, and are subsequently administered to a living animal. The term in vivo administration includes all manipulations performed on cells within an animal. In certain aspects of the present invention, the compositions may be administered either in vitro, ex vivo, or in vivo. An example of in vivo administration includes direct injection of tumors with the instant compositions by intracranial administration to selectively kill tumor cells.

Intratumoral injection, or injection into the tumor vasculature is specifically contemplated for discrete, solid, accessible tumors including tumor exposed during surgery. Local, regional or systemic administration also may be appropriate. For tumors 1.5 to 5 cm in diameter, the injection volume will be 1 to 3 cc, preferably 3 cc. For tumors greater than 5 cm in diameter, the injection volume will be 4 to 10 cc, preferably 5 cc. Multiple injections delivered as single dose comprise about 0.1 to about 0.5 ml volumes, preferable 0.2 ml. The viral particles may advantageously be contacted by administering multiple injections to the tumor, spaced at approximately 1 cm intervals.

In the case of surgical intervention, the present invention may be used preoperatively, to render an inoperable tumor subject to resection. Alternatively, the present invention may be used at the time of surgery, and/or thereafter, to treat residual or metastatic disease. For example, a resected tumor bed may be injected or perfused with a formulation comprising the adenovirus. The perfusion may be continued post-resection, for example, by leaving a catheter implanted at the site of the surgery. Periodic post-surgical treatment also is envisioned.

Continuous administration, preferably via syringe or catheterization, also may be applied where appropriate, for example, where a tumor is excised and the tumor bed is treated to eliminate residual, microscopic disease. Such continuous perfusion may take place for a period from about 1-2 hr, to about 2-6 hr, to about 6-12 hr, to about 12-24 hr, to about 1-2 days, to about 1-2 wk or longer following the initiation of treatment. Generally, the dose of the therapeutic composition via continuous perfusion will be equivalent to that given by a single or multiple injections, adjusted over a period of time during which the perfusion occurs. It is further contemplated that limb perfusion may be used to administer therapeutic compositions of the present invention, particularly in the treatment of melanomas and sarcomas.

Treatment regimens may vary as well, and often depend on tumor type, tumor location, disease progression, and health and age of the patient. Obviously, certain types of tumor will require more aggressive treatment, while at the same time, certain patients cannot tolerate more taxing protocols. The clinician will be best suited to make such decisions based on the known efficacy and toxicity (if any) of the therapeutic formulations.

The adenovirus also may be administered parenterally or intraperitoneally. Solutions of the active compounds as free base or pharmacologically acceptable salts can be prepared in water suitably mixed with a surfactant, such as hydroxypropylcellulose. Dispersions also can be prepared in glycerol, liquid polyethylene glycols, and mixtures thereof and in oils. Under ordinary conditions of storage and use, these preparations contain a preservative to prevent the growth of microorganisms.

The therapeutic compositions of the present invention are advantageously administered in the form of injectable compositions either as liquid solutions or suspensions; solid forms suitable for solution in, or suspension in, liquid prior to injection may also be prepared. These preparations also may be emulsified. A typical composition for such purpose comprises a pharmaceutically acceptable carrier. For instance, the composition may contain 10 mg, 25 mg, 50 mg or up to about 100 mg of human serum albumin per milliliter of phosphate buffered saline. Other pharmaceutically acceptable carriers include aqueous solutions, non-toxic excipients, including salts, preservatives, buffers and the like. Examples of non-aqueous solvents are propylene glycol, polyethylene glycol, vegetable oil and injectable organic esters such as ethyloleate. Aqueous carriers include water, alcoholic/aqueous solutions, saline solutions, parenteral vehicles such as sodium chloride or Ringer's dextrose. Intravenous vehicles include fluid and nutrient replenishers. Preservatives include antimicrobial agents, anti-oxidants, chelating agents and inert gases. The pH and exact concentration of the various components the pharmaceutical composition are adjusted according to well known parameters. When the route is topical, the form may be a cream, ointment, or salve.

In a further embodiment of the invention, an adenovirus or a nucleic acid encoding an adenovirus may be delivered to cells using liposome or immunoliposome delivery. The adenovirus or nucleic acid encoding an adenovirus may be entrapped in a liposome or lipid formulation. Liposomes may be targeted to neoplasic cell by attaching antibodies to the liposome that bind specifically to a cell surface marker on the neoplastic cell. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated is a nucleic acid construct complexed with Lipofectamine (Gibco BRL).

Combination Therapy

Tumor cell resistance to various therapies represents a major problem in clinical oncology. One goal of current cancer research is to find ways to improve the efficacy of chemo- and radiotherapy, as well as other conventional cancer therapies. One way is by combining such traditional therapies with oncolytic adenovirus therapy. Traditional therapy to treat cancers may include removal of all or part of the affected organ, external beam irradiation, xenon arc and argon laser photocoagulation, cryotherapy, immunotherapy and chemotherapy. The choice of treatment is dependent on multiple factors, such as, 1) multifocal or unifocal disease, 2) site and size of the tumor, 3) metastasis of the disease, 4) age of the patient or 5) histopathologic findings (The Genetic Basis of Human Cancer, 1998).

In the context of the present invention, it is contemplated that adenoviral therapy could be used in conjunction with anti-cancer agents, including chemo- or radiotherapeutic intervention, as well as radiodiagnositc techniques. It also may prove effective to combine oncolytic virus therapy with immunotherapy.

A “target” cell contacting a cell cycle modulating agent, such as an oncolytic virus, and at least one other agent may kill cells, inhibit cell growth, inhibit metastasis, inhibit angiogenesis or otherwise reverse or reduce a hyperproliferative phenotype of target cells. These compositions would be provided in a combined amount effective to kill or inhibit proliferation of the target cell. This process may involve contacting the cells with the cell cycle modulator and the agent(s) or factor(s) at the same or different times. This may be achieved by contacting the cell with a single composition or pharmacological formulation that includes both agents, or by contacting the cell with two distinct compositions or formulations, wherein one composition includes the oncolytic adenvirus and the other includes the second agent.

Oncolytic adenoviral therapy may also be combined with other anti-cancer therapies, such as but not limited to immunosuppression. The immunosuppression may be performed as described in WO 96/12406, which is incorporated herein by reference. Examples of immunosuppressive agents include cyclosporine, FK506, cyclophosphamide, and methotrexate.

Alternatively, an oncolytic adenovirus treatment may precede or follow the second agent or treatment by intervals ranging from minutes to weeks. In embodiments where the second agent and oncolytic adenovirus are applied separately to the cell, one would generally ensure that a significant period of time did not expire between each delivery, such that the second agent and cell cycle modulator would still be able to exert an advantageously combined effect on the cell. In such instances, it is contemplated that one would contact the cell with both modalities within about 12-24 hr of each other and, more preferably, within about 6-12 hr of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either cell cycle modulator and/or the second agent will be desired. Various combinations may be employed, where the cell cycle modulator, e.g., oncolytic adenovirus, is “A” and the other agent is “B”, as exemplified below:

A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B

Other combinations are contemplated. Again, to achieve cell killing, both agents are delivered to a cell in a combined amount effective to kill the cell.

Agents or factors suitable for use in a combined therapy are any anti-angiogenic agent and/or any chemical compound or treatment method with anticancer activity; therefore, the term “anticancer agent” that is used throughout this application refers to an agent with anticancer activity. These compounds or methods include alkylating agents, topoisomerase I inhibitors, topoisomerase II inhibitors, RNA/DNA antimetabolites, DNA antimetabolites, antimitotic agents, as well as DNA damaging agents, which induce DNA damage when applied to a cell. In particular aspects the second agent is a S-phase specific anti-cancer agent.

Examples of chemotherapy drugs and pro-drugs include, CPT11, temozolomide, platin compounds and pro-drugs such as 5-FC. Examples of alkylating agents include, inter alia, chloroambucil, cis-platinum, cyclodisone, flurodopan, methyl CCNU, piperazinedione, teroxirone. Topoisomerase I inhibitors encompass compounds such as camptothecin and camptothecin derivatives, as well as morpholinodoxorubicin. Doxorubicin, pyrazoloacridine, mitoxantrone, and rubidazone are illustrations of topoisomerase II inhibitors. RNA/DNA antimetabolites include L-alanosine, 5-fluoraouracil, aminopterin derivatives, methotrexate, and pyrazofurin; while the DNA antimetabolite group encompasses, for example, ara-C, guanozole, hydroxyurea, thiopurine. Typical antimitotic agents are colchicine, rhizoxin, taxol, and vinblastine sulfate. Other agents and factors include radiation and waves that induce DNA damage such as, γ-irradiation, X-rays, UV-irradiation, microwaves, electronic emissions, and the like. A variety of anti-cancer agents, also described as “chemotherapeutic agents,” function to induce DNA damage, all of which are intended to be of use in the combined treatment methods disclosed herein. Chemotherapeutic agents contemplated to be of use, include, e.g., adriamycin, bleomycin, 5-fluorouracil (5-FU), etoposide (VP-16), camptothecin, actinomycin-D, mitomycin C, cisplatin (CDDP), podophyllotoxin, verapamil, and even hydrogen peroxide. The invention also encompasses the use of a combination of one or more DNA damaging agents, whether radiation-based or actual compounds, such as the use of X-rays with cisplatin or the use of cisplatin with etoposide.

In treating pre-cancer or cancer according to the invention, one would contact the cells of a precancerous lesion or tumor cells with an agent in addition to the cell cycle modulator, e.g., oncolytic adenovirus. This may be achieved by irradiating the localized tumor site with radiation such as X-rays, UV-light, γ-rays or even microwaves. Alternatively, the cells may be contacted with the agent by administering to the subject a therapeutically effective amount of a pharmaceutical composition comprising a compound such as, adriamycin, bleomycin, 5-fluorouracil, etoposide, camptothecin, actinomycin-D, mitomycin C, podophyllotoxin, verapamil, or more preferably, cisplatin. The agent may be prepared and used as a combined therapeutic composition, or kit, by combining it with a cell cycle modulator.

Agents that directly cross-link nucleic acids, specifically DNA, are envisaged to facilitate DNA damage leading to a synergistic, anti-neoplastic combination with an oncolytic adenovirus. Cisplatinum agents such as cisplatin, and other DNA alkylating agents may be used. Cisplatin has been widely used to treat cancer, with efficacious doses used in clinical applications of 20 mg/m² for 5 days every three weeks for a total of three courses. Cisplatin is not absorbed orally and must therefore be delivered via injection intravenously, subcutaneously, intratumorally or intraperitoneally. Bleomycin and mitomycin C are other anticancer agents that are administered by injection intravenously, subcutaneously, intratumorally or intraperitoneally. A typical dose of bleomycin is 10 mg/m², while such a dose for mitomycin C is 20 mg/m².

Agents that damage DNA also include compounds that interfere with DNA replication, mitosis and chromosomal segregation. Such chemotherapeutic compounds include adriamycin, also known as doxorubicin, etoposide, verapamil, podophyllotoxin, and the like. Widely used in a clinical setting for the treatment of neoplasms, these compounds are administered through bolus injections intravenously at doses ranging from 25-75 mg/m² at 21 day intervals for adriamycin, to 35-50 mg/m² for etoposide intravenously or double the intravenous dose orally.

Agents that disrupt the synthesis and fidelity of nucleic acid precursors and subunits also lead to DNA damage. As such a number of nucleic acid precursors have been developed. Particularly useful are agents that have undergone extensive testing and are readily available. As such, agents such as 5-fluorouracil (5-FU), are preferentially used by neoplastic tissue, making this agent particularly useful for targeting to neoplastic cells. Although quite toxic, 5-FU, is applicable in a wide range of carriers, including topical, however intravenous administration with doses ranging from 3 to 15 mg/kg/day being commonly used or as alternative 5-FC may be administered and converted in a target tissue or target cell.

Other factors that cause DNA damage and have been used extensively include what are commonly known as γ-rays, X-rays, and/or the directed delivery of radioisotopes to tumor cells. Other forms of DNA damaging factors are also contemplated such as microwaves and UV-irradiation. It is most likely that all of these factors effect a broad range of damage DNA, on the precursors of DNA, the replication and repair of DNA, and the assembly and maintenance of chromosomes. Dosage ranges for X-rays range from daily doses of 50 to 200 roentgens for prolonged periods of time (3 to 4 weeks), to single doses of 2000 to 6000 roentgens. Dosage ranges for radioisotopes vary widely, and depend on the half-life of the isotope, the strength and type of radiation emitted, and the uptake by the neoplastic cells.

Immunotherapy may be used as part of a combined therapy, in conjunction with mutant oncolytic virus-mediated therapy. The general approach for combined therapy is discussed below. Generally, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155. Antibodies specific for CAR, integrin or other cell surface molecules, may be used to identify cells that the adenovirus could infect well. CAR is an adenovirus receptor protein. The penton base of adenovirus mediates viral attachment to integrin receptors and particle internalization.

The skilled artisan is directed to “Remington's Pharmaceutical Sciences” 15th Edition, 1980. Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

The inventors propose that local, regional delivery of a cell cycle modulator, e.g., oncolytic adenovirus to patients with retinoblastoma-linked cancers, pre-cancers, or hyperproliferative conditions will be a very efficient method for delivering a therapeutically effective gene. Similarly, the chemo- or radiotherapy may be directed to a particular, affected region of the subjects body. Alternatively, systemic delivery of expression construct and/or the agent may be appropriate in certain circumstances, for example, where extensive metastasis has occurred.

In addition to combining cell cycle modulator therapies with chemo- and radiotherapies, it also is contemplated that combination with gene therapies will be advantageous. For example, the inventive methods in combination with the targeting of p53 at the same time may produce an improved anti-cancer treatment. Any tumor-related gene or nucleic acid encoding a polypeptide conceivably can be targeted in this manner, for example, p21, Rb, APC, DCC, NF-1, NF-2, BCRA2, p16, FHIT, WT-1, MEN-I, MEN-II, BRCA1, VHL, FCC, MCC, ras, myc, neu, raf, erb, src, fms, jun, trk, ret, gsp, hst, bcl and abl.

It is further contemplated that the therapies described above may be implemented in combination with all types of surgery. Approximately 60% of persons with cancer will undergo surgery of some type, which includes preventative, diagnostic or staging, curative and palliative surgery. These types of surgery may be used in conjunction with other therapies, such as oncolytic adenovirus therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs surgery). It is further contemplated that the present invention may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection, systemic administration, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well. Furthermore, in treatments involving more than a single treatment type (i.e., construct, anticancer agent and surgery), the time between such treatment types may be about 1, 2, 3, 4, 5, 6, 7, 8, 9, 10, 11, 12, 13, 14, 15, 16, 17, 18, 19, 20, 21, 22, 23, or about 24 hours apart; about 1, 2, 3, 4, 5, 6, or 7 days apart; about 1, 2, 3, 4, or 5 weeks apart; and about 1, 2, 3, 4, 5, 6, 7, 8, 9, 10, 11, or 12 months apart, or more.

It also should be pointed out that any of the foregoing therapies may prove useful by themselves. In this regard, reference to chemotherapeutics and non- mutant oncolytic virus therapy in combination also should be read as a contemplation that these approaches may be employed separately.

A tumor may be biopsied and the above tests performed upon it to determine whether the cells have a functional Rb pathway or to assess the proportion of cells in particular phase of the cell cycle. An example of a biopsy protocol is as follows. The stereotactic biopsy is the precise introduction of a metal probe into the brain tumor, cutting a small piece of the brain tumor, and removing it so that it can be examined under the microscope. The patient is transported to the MRI or CAT scan suite, and the frame is attached to the scalp under local anesthesia. The “pins” of the frame attach to the outer table of the skull for rigid fixation (frame will not and can not move from that point forward until completion of the biopsy). The scan (MRI or CT) is obtained. The neurosurgeon examines the scan and determines the safest trajectory or path to the target. This means avoiding critical structures. The spatial co-ordinates of the target are determined, and the optimal path is elected. The biopsy is carried out under general anesthesia. A small incision is created over the entry point, and a small hole is drilled through the skull. The “dura” is perforated, and the biopsy probe is introduced slowly to the target. The biopsy specimen is withdrawn and placed in preservative fluid for examination under the microscope. Often the pathologist is present in the biopsy suite so that a rapid determination of the success of the biopsy can be made.

### EXAMPLES

The following examples are included to demonstrate preferred embodiments of the invention. It should be appreciated by those skilled in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### Example 1

Materials and Methods

Cell lines, adenoviral constructs, and infection conditions. The human glioma cell lines U-87 MG and U-251 MG were purchased from the American Type Culture Collection (Manassas, Va.). Cells were maintained in DMEM/F-12 (1:1 v/v; The University of Texas M. D. Anderson Cancer Center Media Core Facility) supplemented with 10% FCS and 1% antibiotic/antimycotic agent (Life Technologies, Grand Island, N.Y.) in a humidified atmosphere containing 5% CO₂ at 37° C. The replication-selective adenovirus Delta-24 has been described previously (Fueyo et al., 2003). This construct has a 24-bp deletion of the E1a region (nucleotides 923-946, both included), corresponding to amino acids L₁₂₂TCHEAGF₁₂₉, a region required for Rb protein binding. As controls, wild-type adenovirus Ad300 was used (Jones and Shenk, 1979), Delta-24 virus inactivated by UV light (UVi Delta-24; inactivated by exposure to seven cycles of 125 J UV light), and mock infections with culture medium.

The glioma cells were infected as described previously (Fueyo et al., 2000). Briefly, the viral stocks were diluted to the indicated multiplicities of infection (MOI; plaque-forming units per cell), added to cell monolayers (0.5 mL/60-mm dish or 5 mL/100-mm dish), and incubated at 37° C. for 30 minutes with brief agitation every 5 minutes. After this, the necessary amount of culture medium was added and the cells were returned to the incubator for the prescribed times. Drugs. CPT-11 was kindly provided by Pharmacia Corp. (Kalamazoo, Mich.). Stocks of 20 mg/mL in aqueous solution were kept at 4° C.

Western blot analyses. Glioma cells were infected with 50 MOIs of Delta-24, wild-type Ad300, or UVi Delta-24 or were mock infected. Total cell lysates were prepared 20 hours after infection by incubating the cells in radioimmunoprecipitation assay buffer [150 mmol/L NaCl, 1% Triton X-100, 1% sodium deoxycholate, 0.1% SDS, 20 mmol/L EDTA, and 50 mmol/L Tris (pH 7.4)] for 1 hour at 4° C. Protein (50 μg) from each sample was subjected to 10% SDS-Trisglycine gel electrophoresis and transferred to a nitrocellulose membrane (Schleicher & Schuell, Inc., Keene, N.H.). The membrane was blocked with Blotto-Tween [3% nonfat milk, 0.05% Tween 20, 0.9% NaCl, and 50 mmol/L Tris (pH 7.5)] and incubated with rabbit anti-human topoisomerase I serum (dilution 1:2,500; TopoGEN, Inc., Columbus, Ohio); mouse anti-human actin monoclonal antibody IgG (dilution 1:3,000; Amersham Corp., Arlington Heights, Ill.) was used as a loading control. The secondary antibodies were horseradish peroxidase—conjugated donkey anti-rabbit and goat anti-mouse IgG (Amersham). The membranes were developed according to Amersham enhanced chemiluminescence protocol.

DNA topoisomerase I activity. The activity of topoisomerase I was determined by measuring the relaxation of supercoiled Escherichia coli DNA (pBR322) using the topoisomerase I assay kit (TopoGEN) essentially according to the method of Liu and Miller (1981). First, 2×10⁶ U-87 MG or U-251 MG cells were seeded, and 24 hours later, the cells were infected with Delta-24 or UVi Delta-24 at a MOI of 50. Twenty hours after infection, topoisomerase I was extracted as described previously (Trask and Muller, 1983). Topoisomerase I activity was determined following the instructions that came with the assay kit. Briefly, the reaction mixtures used contained supercoiled (form I) plasmid substrate DNA, nuclear extract (5.0 μg/mL protein), and the assay buffer. Positive control samples contained topoisomerase 1 (5 units). The reaction mixtures were incubated at 37° C. for 30 minutes, and the reactions were terminated by adding 5 μL stop buffer/gel loading buffer. Proteinase K (Qiagen, Valencia, Calif.) was added to a concentration of 50 μg/mL, and the mixture was digested for 60 minutes at 37° C. Samples were loaded onto a 1% agarose gel and electrophoresed overnight at room temperature in a running buffer of Tris-acetate EDTA with chloroquine (0.2 μg/mL; Sigma-Aldrich, St. Louis, Mo.). The gel was stained with 0.5 μg/mL ethidium bromide.

Cell cycle analysis. The DNA content was measured in samples of 10⁶ cells that had been infected with 10 MOIs of Delta-24 or UVi Delta-24 or had been mock infected. Forty-eight hours later, cultures were treated with CPT-11 (4 μmol/L) or vehicle. Cells were trypsinized 3 to 5 days after drug treatment, fixed in 70% ice-cold ethanol, and incubated with propidium iodide (5 μg/mL) and RNase A (1 μg/mL) for 20 minutes at 37° C. All DNA content measurements were done with an EPICS XL-MCL cytometer (Coulter Corp., Hialeah, Fla.) equipped with an air-cooled argon ion laser emitting 488 nm at 15 mW. A multicycle program (Phoenix Flow System; Phoenix Controls Corp., San Diego, Calif.) was used for data analysis. Cell viability assays. The chemosensitivity of the treated glioma cells was assessed by using the tetrazolium salt 3-(4,5-dimethylthiazol-2-yl)-2,5-diphenyltetrazolium bromide (Sigma-Aldrich) to measure cell viability. For this assay, 2×10³ cells per well were seeded in 96-well microtiter plates and infected 24 hours later with Delta-24 (at 1, 2.5, 5, or 10 MOs) or Uvi Delta-24 (10 MOs) or were mock infected. Forty-eight hours after adenoviral treatment, the cells were treated with various concentrations of CPT-11. Triplicate wells were used for each condition. Sixteen wells seeded with untreated glioma cells were used as a viability control, and 16 wells containing only complete medium were used as a control for nonspecific dye reduction. Medium was removed 72 hours after drug treatment, and 100 μL 3-(4,5-dimethylthiazol-2-yl)-2,5-diphenyltetrazolium bromide solution (1 mg/mL) was added to each well. The plates were then incubated for an additional 4 hours and then read on a Spectramax 190 microplate reader (Molecular Devices, Sunnyvale, Calif.) at a test wavelength of 570 nm.

Viral replication assays. U-87 MG and U-251 MG human glioma cells were seeded at a density of 5×10⁴ per well in six-well plates and infected 20 hours later with Delta-24 or Uvi Delta-24 at a MOI of 1. CPT-11 (5 μmol/L) was added 48 hours later. Three days after drug treatment, cells were scraped into culture medium and lysed with three cycles of freezing and thawing. The TCID₅₀ method was used to determine the final viral titration as described previously (Fueyo et al., 2003). Briefly, the cell lysates were clarified by centrifugation and the supernatants were serially diluted in medium for infecting 293 cells in 96-well plates. The cells were analyzed for cytopathic effect 10 days after infection. Final titers were determined as plaque-forming units (pfu) using the validation method developed by Quantum Biotechnology (Carlsbad, Calif.).

Animal model. To assess the potential therapeutic relevance of the findings from the in vitro studies, we tested the Delta-24/CPT-11 sequential treatment in an in vivo model of a human glioma xenograft implanted intracranially. The U-87 MG cell line was selected as an exemplary cell line because it produces gliomas in nude mice with highly predictable growth kinetics and well-characterized pathologic features (Lal et al., 2000). To perform a reliable multiple-dose experiment, an implantable guide-screw system was developed that allows for multiple, precise intratumoral administrations of a therapeutic agent(s) (Lal et al., 2000) and has been validated for testing the antiglioma effect of Delta-24 (Fueyo et al., 2003). In this study, 5×10⁵ cells of the U-87 MG human glioma cell line were engrafted in the caudate nucleus of athymic mice (Harlan- Sprague-Dawley, Inc., Indianapolis, Ind.). On day 3 after cell implantation, animals were treated with a single intratumoral injection of Delta-24 or UVi Delta-24 (1.5×10⁸ viral particles in 5 μL). CPT-11 was given on days 7, 12, and 20 after cell implantation (5 mg/kg i.p.). Animals showing general or local symptoms of toxicity were sacrificed, and the surviving animals were sacrificed 110 days after engraftment. Brains were fixed in 4% formaldehyde for 24 hours and embedded in paraffin. H&E-stained slides were analyzed for evidence of tumor, necrosis, and viral nuclear inclusions. Animal studies were done in the veterinary facilities of M.D. Anderson Cancer Center in accordance with institutional guidelines.

Statistical analyses. For the in vitro experiments, statistical analyses were done using a two-tailed Student's t test. Data are mean F SD. The in vivo anticancer effect of different treatments was assessed by plotting survival curves according to the Kaplan-Meier method, and groups were compared using the log-rank test.

Results

Delta-24 infection enhanced expression and activity of topoisomerase I. The inventors investigate whether Delta-24 adenovirus could sensitize glioma cells to the camptothecin analogue CPT-11 by up-regulation of topoisomerase I expression. The expression of topoisomerase I was assessed in the U-87 MG and U-251 MG human glioma cells after infection with Delta-24. These two cell lines were selected because they were used previously to characterize the antiglioma effect of Delta-24 (Fueyo et al., 2000). Western blot analysis showed that endogenous topoisomerase I was expressed at a low level in both glioma cell lines. However, treatment with Delta-24 resulted in at least a 4-fold increase in topoisomerase levels in both cell lines compared with mock-infected or UVi Delta-24-infected cells. Cells infected with the wild-type adenovirus exhibited an increase in topoisomerase I expression similar to that seen in the Delta-24- infected cells.

The inventors determined whether Delta-24 infection resulted in increased topoisomerase I activity in glioma cells in culture. A plasmid DNA used as a template for the topoisomerase I reaction incubated with UVi Delta-24-infected nuclear extracts appeared predominantly in the supercoiled form, similar to the finding in the control cells containing the form I DNA plasmid without topoisomerase I. In addition, Delta-24-infected nuclear extracts from both glioma cultures displayed a topoisomerase I activity that caused the plasmidic DNA to relax comparable with the finding in the topoisomerase I-treated positive controls. Taken together, these observations indicate that infection with the Delta-24 adenovirus increases topoisomerase I protein levels and activity.

Cell cycle profile of Delta-24- and CPT-11-treated cells. Previous data showed that Delta-24 infections cause cells to accumulate in the S phase of the cell cycle (Fueyo et al., 2000). In this study, U-87 MG and U-251 MG human glioma cells were infected with Delta-24 adenovirus and treated 2 days later with CPT-11. Cells were then collected and their DNA content was examined by flow cytometry. As expected, the accumulation of Delta-24-infected cells in the S phase of the cell cycle was striking (>70% of the cells in culture) and statistically significant in comparison with the control cells infected with UVi Delta-24 (P<0.001; Table 1). It was also shown that treatment with CPT-11 resulted in an accumulation of cells in the G2-M phase (>55% of the cells in culture; P<0.001, compared with vehicle-treated cells). The inventors investigated the effect of the combination of the Delta-24 adenovirus and CPT-11 on cell cycle progression. Cells infected with Delta-24 and then treated 48 hours later with CPT-11 showed an overrepresentation of cells in the S phase (>65% of the cells in culture) with a dramatic decrease in the G2-M population (<20% of the cells in culture). Thus, cells treated with a combined regimen exhibited a cell cycle profile similar to that of cells treated with Delta-24 alone.

These data indicate that Delta-24 infection overrides the G2-M arrest induced by CPT-11 and maintains a large population of cells in the S phase, suggesting that Delta-24-infected cells are likely to be particularly susceptible to S-phase-based chemotherapeutic agents.

Effect of CPT-11 and Delta-24 on proliferation of human glioma cells. The inventors ascertained the sensitivity of U-87 MG and U-251 MG glioma cells to CPT-11. In both cell lines, CPT-11 inhibited cell proliferation in a concentration dependent fashion as assessed by the 3-(4,5-dimethylthiazol-2-yl)-2,5-diphenyltetrazolium bromide assay. In contrast, no significant inhibition was seen in control cells treated with equivalent concentrations of vehicle in the absence of CPT-11 (data not shown). After this, the effect of the sequential administration of Delta-24 combined with several different CPT-11 concentrations was assessed. For these experiments, the inventors designed a treatment schedule based on the hypothetical mechanism of the Delta-24-mediated potentiation of the drug effect (i.e., induction of topoisomerase I) and previous data indicating that the greatest accumulation of cells in the S phase occurs within 48 hours of Delta-24 infection (Fueyo et al., 2000). Thus, cells were infected with Delta-24 at a range of 1 to 10 MOIs, and CPT-11 was added 48 hours later. The IC₅₀ dose of CPT-11 decreased from 3.4 μmol/L in U-87 MG cells infected with UVi Delta-24 to 1.5 μmol/L in Delta-24-infected cells infected at a dose of 10 MOIs (P<0.001) and from 7.2 μmol/L in UVi Delta-24-infected U-251 MG cells to 1 Amol/L in Delta-24-infected cells infected with 10 MOIs (P<0.001). The IC₅₀ for CPT-11 was modified significantly (to 2.5 μmol/L) in both U-87 MG and U-251 MG cells infected with 2 MOIs of Delta-24.

Importantly, in an independent set of experiments, Delta-24 was tested as a potentiator of the CPT-11-mediated cytotoxicity in glioma cultures. In this experiment in which low doses of both Delta-24 (2 MOs) and CPT-11 (2.5 μmol/L) were used, it was observed that the effect of the combination of the two agents exceeded the total effect of the two when given alone in both U-87 MG and U-251 MG cells. Sequential administration of Delta-24 and CPT-11 did not modify the replication capability of the adenovirus. Viral replication assays done after the combined treatment of glioma cells with Delta-24 and CPT-11 showed that there was no significant modification in the replicative phenotype of the oncolytic adenovirus when it was combined with the topoisomerase I inhibitor under the conditions used in this experiment. Specifically, the resulting viral titers after both treatments differed by only 0.13±0.37 and 0.63±0.45 orders of magnitude in the U-87 MG and U-251 MG cells, respectively. These results show that Delta-24 replicated with a similar efficiency whether given singly or in combination with CPT-11 (P>0.1; double-sided t test). This indicated that the anticancer effect observed in cells treated with Delta-24 and CPT-11 could be the result of the Delta-24-mediated potentiation of the effect of CPT-11.

Combined antiglioma effect of Delta-24 and CPT-11 in vivo. To assess the potential therapeutic relevance of the findings from the in vitro studies, the Delta-24/CPT-11 sequential treatment was tested in an in vivo model of a human glioma xenograft implanted intracranially that had been validated for testing the antiglioma effect of Delta-24 (Fueyo et al., 2003). On day 3 after U-87 MG cell implantation, animals were treated with a single intratumoral injection of Delta-24 or UVi Delta-24 (1.5×10⁸ viral particles in 5 μL). CPT-11 was given on days 7, 12, and 20 after cell implantation (5 mg/kg i.p.). The median survival was 27 days in the control group of animals (treated with vehicle plus UVi Delta-24), and all these animals died by day 32. Treatment with CPT-11 (plus UVi Delta-24) or a single dose of Delta-24 (plus vehicle) extended the survival by an average of 4 and 8 days, respectively (P=0.001 and P<0.0001, respectively, compared with vehicle-treated animals). The combination treatment consisting of Delta-24 followed by CPT-11 resulted in the most substantial increase in animal survival (median overall survival of 42 days). In addition, the overall survival of the animals treated with the combined therapy differed significantly from that in animals treated with either agent alone (P<0.005, log-rank test), as did differences in the 60-day survival rate (P<0.012, Fisher's test). Of further relevance, there were no long-term survivors among the animals receiving a single-treatment regimen; however, 7 of 31 (22.5%) animals treated with the combination of Delta-24 and CPT-11 survived >3 months after tumor implantation without showing any sign of neurologic distress. Examination of the brains of the animals showed that all animals that died had evidence of intracranial tumor. In contrast, microscopic examination of the brains of the longterm surviving animals that received the combination treatment showed complete tumor regression. In these animals, however, sequelae of the tumors were identified, including dystrophic calcification and microcyst formation, at the tumor implantation site in the right caudate nucleus. Immunohistochemical analyses of the brains of the long-term survivors using both anti-E1A and anti-hexon antibodies failed to detect viral particles (data not shown). Further, E1A expression or signs of inflammation in the normal brain tissue was not observed.

### Example 2

Material and Methods

Cell Lines and Culture Conditions. The glioma cell lines U-251 MG and U-87 MG were obtained from the American Type Culture Collection (ATCC). Cell lines were maintained in Dulbecco's modified Eagle/F 12 medium (DMEM/F 12) (1:1, vol/vol) supplemented with 10% fetal bovine serum in a humidified atmosphere containing 5% CO₂ at 37° C. Normal human astrocytes (NHAs) were purchased from Clonetics/BioWhittaker. NHA cultures were maintained in astrocyte growth medium from an AGM-Astrocyte Medium BulletKit obtained from Clonetics/BioWhittaker. For serum starvation conditions, we grew NHAs at a low density (2×10⁴/per well in six-well plate) in the kit's medium with 0.5% fetal bovine serum and no growth supplements. These culture conditions inhibited cell growth without evidence of cell death.

Adenovirus Construction and Infection. Contruction of WT-RGD, Delta-24 and Delta-24-RGD have been previously described (Pasqualini, 1997; Fueyo, 2000; Suzuki, 2001; Fueyo, 2003). Wild-type adenovirus Ad300 (Jones, 1978), ICOVIR-5 inactivated by UV light and mock-infected cells (i.e., with DMEM/F12) were used as controls.

The human E2F-1 promoter was synthesised from normal human PBMC by PCR using oligonucleotides that amplify from −218 to +51 of E2F-1 sequence and subcloned into pGL3-plasmid (Promega) to generate pGL3-E2F. From this plasmid, the E2F promoter was subcloned into a pXC1-Delta-24 (Fueyo, 2000) modified to contain a cloning site linker inserted between nt 348 and nt 522 of Ad5 genome. The resulting plasmid was named pE2F-Delta-24. The modified E1a region of this plasmid was introduced into pShuttle (He, 1998) to yield pShuttle-E2F-Delta-24.

The human DM-1 insulator genomic DNA was obtained from normal human peripheral blood mononuclear cells by PCR using oligonucleotides that amplify from nt 13006 to nt 13474 of DM-1 locus sequence (GenBank accession no. L08835). This region contains the CTCF-binding sites and the CTG repeats responsible for the insulator activity of the DM-1 locus (Filippova, 2001). The PCR primers were designed to incorporate Xho I flanking sites. DM-1 insulator was cut with XhoI and subcloned into the XhoI site of pShuttle-E2F-Delta-24 to obtain pShuttle-DM-E2F-Delta-24.

To insert the Kozak sequence before E1A, a Kpn1 fragment from pShuttle-DM-E2F-Delta-24 containing the E2F promoter and E1A was subcloned into pGEM-3Z (Promega) and this plasmid was used to replace the E1A translation start site using oligonucleotides with the Kozak sequence. The Kpn1 fragment containing the E2F-E1A modified with the Kozak sequence was returned to pShuttle-DM-E2F-Delta-24 to obtain pShuttle-DM-E2F-KDelta-24.

Finally pShuttle-DM-E2F-Delta-24 was recombined with pVK503 that contains complete Ad5 genome with RGD-modified fiber (Dmitriev, 1998) by homologous recombination to construct pICOVIR5. Virus ICOVIR-5 was obtained after digestion of this plasmid with Pacl and transfection into HEK293. ICOVIR-5 was then plaque-purified and amplified in A549 cells and purified using a two-step CsCl gradient centrifugation. Virus genomic structure was verified by restriction analysis. Sequencing of DM-1 insulator, E2F promoter, Kozak sequence, E1A-Delta-24 deletion and RGD fiber was carried out using oligonucleotides DM1-Up (5′-GGGCAGATGGAGGGCCTTTTATTC-3′), E2F-Up (5′-GTGTTACTCATAGCGCGTAA-3′), Delta-24-down (5′-CCTCCGGTGATAATGACAAG-3′) and FiberUp (5′-CAAACGCTGTTGGATTTATG-3′).

Cell Viability Assay. Human glioma cells were seeded (at 10⁵ cells per well) in DMEM/F12 medium in six-well plates and allowed to grow for 20 hours at 37° C. Cells were then infected with Ad300, WT-RGD, Delta-24-RGD and ICOVIR-5 and or UV-inactivated of each of the former virus at MOI of 0.1, 1, 5 and 10 at 37° C. for 30 minutes. Experiments were concluded when an MOI of 10 for one of the adenoviral constructs produced a cytopathic effect of more than 50%. The cell monolayers were then washed twice with PBS and were fixed and stained with 0.1% crystal violet in 20% ethanol. Excess dye was removed by rinsing several times with water. MTT experiments were performed to quantify cell viability, as previously described (as described by Mossman). Briefly, cultures were infected with the adenoviral constructs at MOIs of 0, 0. 1, 1, 5 and 10 and then after 7 days the experiments were stopped.

Cell Cycle Analysis. Cell-cycle phase distribution was analyzed by measuring DNA content, as described previously (Gomez-Manzano, 1997). Cell samples were collected at different time points after infection with WT-RGD, Delta 24-RGD or ICOVIR-5.

Luciferase Assays. Cells were seeded at a density of 3×10⁴ cells/well in 24-well dishes and cultured for 24 h. Cells were then transfected with 250 ng of E2F1 reporter construct (Johnson, 1994) by using FuGENE 6 transfection reagent (Roche Diagnostics Corp.). One hour after transfection, cells were infected with Mock, UV-inactivated WT-RGD, WT-RGD, Delta-24-RGD, ICOVIR-5, Ad-β-Gal, at 50 MOIs. Cells were harvested 24 h after treatment, and reporter activity was measured using the Dual Luciferase assay (Promega). Luciferase activity from untreated control cells was used for the background signal. Transfections were normalized for efficiency using pRL-CMV (Promega) and expressed as folds of induction relative to mock-treated cells (arbitrary value of 1).

Infection with Exogenous Wild-Type Rb or p21. The Rb and p21 adenoviruses used in this study and their infectivity have been previously described (Fueyo, 1998)(Gomez-Manzano, 1997). Briefly, after U251 MG and U87 MG were seeded in DMEM/F12 medium in six-well plates, the cultures were infected with replication-deficient adenoviral vectors expressing either Rb or p21 or with the control adenoviral vector Ad5CMV-pA (with an empty expression cassette) at an MOI of 80 at 37° C. for 30 minutes. Seventy-two hours later, the cultures were treated with either WT-RGD, ICOVIR-52 or both virus UV-inactivated at an MOI of 10 at 37° C. for 30 minutes. Cell viability was monitored daily and was quantified using the trypan blue exclusion test.

Immunoblotting. For immunoblotting assays cells were lysed in RIPA buffer for 30 min on ice, and then passed through a 23-gauge needle. Membranes were incubated with the following antibodies: E2F1 (KH-95), β-actin, E1A, Fiber, Tubulin, (C-11; Santa Cruz). pRb, p21, GFAP. The membranes were developed according to Amersham's enhanced chemiluminiscence (ECL) protocol. Protein expression was quantified by densitometry analysis on a Macintosh computer using the public domain NIH Image program (developed at the U.S. National Institutes of Health and available on the Internet at rsb.info.nih.gov/nih-image/).

Chromatin immunoprecipitation (ChIP) assays. For the in vitro ChIP assay U251 MG or U87 MG or NHA were infected with ICOVIR-5, Delta-24-RGD, ICOVIR-5 UV-inactivated, or were mock-treated for 24 hrs. Cells were then fixed with 1% formaldehyde for 10 min in at 37° C. Fixed cells were washed twice with PBS containing a mixture of protease inhibitors (Sigma), and suspended in 200 μl lysis buffer (1%SDS, 10 mM EDTA, 50 mM Tris-HCl (pH 8.1) and protease inhibitors (Sigma). Chromatin was sheared by sonicating 5 times for 10 s at a setting of 4 using a 60 Sonic Dismembrator (Fischer) followed by centrifugation for 10 min at 14000 rpm. Twenty μl of the resulting supernatant was set aside as input chromatin. The subsequent IP and extraction methods were carried out using a commercially available ChIP assay kit (Upstate Biotechnology) following the manufacturer's instruction. E2F1 (KH-95), or mouse IgG antibodies (Santa Cruz) were used to immunoprecipitate the cross-linked chromatin. The following primers were used to amplify a 272-bp fragment in the E2F1 promoter and the adjacent viral genome: 5′-TGTCTGTCCCCACCTAGGAC-3′ and 5′-GCGGTTCCTATTGGCTTTAAC-3′. E2 primers were designed to amplify a 52-bp fragment in the E2 promoter containing two binding sites for E2F1: 5′-TCGAACAAAAGCGCGAATTTAA-3′ AND 5′-TTAAACTCTTTCCCGCGCTTTGATCAGT-3′.

For the animal ChIP assay, the brain from animals previously engrafted with the U87 MG glioma cell line (5×10⁵) and treated with PBS, 300, Delta-24-RGD or ICOVIR-5 were extracted. Brains were then fixed with 1% formaldehyde for 15 min in at RT. Fixed brains were washed twice with PBS containing a mixture of protease inhibitors (Sigma) and 01M glycine. Then suspended in 200 μl lysis buffer (1%SDS, 10 mM EDTA, 50 mM Tris-HCl (pH 8.1) and protease inhibitors (Sigma) and tissue was homogenized with a manual homogenizer. Chromatin was sheared by sonicating 4 times for 10s at a setting of 8 using a 60 Sonic Dismembrator (Fischer) followed by centrifugation for 10 min at 14000 rpm. Twenty μl of the resulting supernatant was set aside as input chromatin.

Taqman analysis. Quantitative-PCR analysis will be performed on a Chromo 4 sequence detection system (Bio-Rad). Quantitative detection of specific nucleotide sequences was based upon the fluorogenic 5′ nuclease assay. The developed assays typically had primer and probe concentrations of 300 nM and 200 nM, respectively. Typically the probes were modified at their 5′ends with the 6-FAM fluorophore group and with the TAMRA quencher at their 3′-ends. Assays were optimized to >90% efficiency according to standard procedures using the enzymes, nucleotides and the ROX passive reference of the TaqMan master mixture (ABI). RNA was isolated with the RNAeasy kit (Qiagen). All measurements were performed with 1 μg of isolated total RNA, at least in duplicate. RNA was reverse transcribed into cDNA in a 25-μL reaction containing 1×PCR buffer (ABI), 7.5 mmol/L MgCl₂, 1 mmol/L each deoxynucleotide triphosphate, 5 μmol/L hexamers (Life Technologies), RNase inhibitor (0.4 units/μL; Roche), and MuLvRT (2.5 units/μL; Life Technologies). Reactions were incubated at 25° C. for 10 minutes, 48° C. for 40 minutes, and 95° C. for 5 minutes. To show that cDNA synthesis from RNAs was quantitative, input RNA concentrations were varied (500, 250, 125, and 62.5 ng), and 5 μL were used in triplicate in real-time amplification. For normalization, cDNA equivalent to input RNA was measured in duplicate for β-actin transcripts by real time PCR (Hs99999903_ml, ABI). Each gene transcript measurement was also tested on a serial dilution of one sample to confirm >90% efficiency of the reaction. Primer and probe sequences were selected for a Tm of 60° C. and 70° C., respectively, and keeping the amplicon size as short as possible. For the detection of adenoviral E1A mRNA transcripts, the reverse primer E1A-R: 5′-TCGGGCGTCTCAGGATAGC-3′ and probe E1A-P: 5′-6FAMAGCCTGCAAGACCTACCCGCCGT-TAMRA-3′ were used with E1A-F: 5′-GAGGATGAAGAGGGTCCTGTGT-3′ For the detection of adenoviral fiber, the common forward primer fiber-F: 5′-CGGCCTCCGAACGGTACT-3′ and probe fiber-P: 5′-6FAMTCTCGAGAAAGGCGTCTAACCAGTCACAGT-TAMRA-3′ were used in combination with the Fiber-specific reverse primer Fiber-R: 5′-TCTTGCGCGCTTCATCTTG-3′. The PCR profile would be perform as follows: 10 minutes at 95° C. for 1 cycle; 15 seconds at 95° C., 1 minute at 60° C. for 40 cycles (Johnson, 2002).

Animal Studies. U87 MG human glioma cells (5×10⁵) were engrafted into the caudate nucleus of athymic mice using a guide-screw system, as previously described (Lal, 2000). The inventors performed three independent experiments using 10 animals per group in each experiment. On days 3, 5, and 7 after implantation of tumor cells, animals were treated with 5 μl intratumoral injections of ICOVIR-5, PBS or adenovirus control (all 3×10⁸ pfu/ml). Animals showing general or local symptoms of toxicity were killed. Surviving animals were killed 140 days after tumor implantation. Brains were then removed, fixed in 4% formaldehyde for 24 hours at room temperature, and embedded in paraffin. Hematoxylin-and-eosin-stained sections were evaluated for evidence of tumor, necrosis, and viral nuclear inclusions. The largest (a) and the smallest (b) diameters of the tumors were measures, and these measurements were used in the calculation of tumor volume using the formula a×b2×0.4 (Attia, 1966). For the combination studies with chemotherapy, on days 3, 5, and 7 after implantation of tumor cells, animals were treated with 5 μl intratumoral injections of ICOVIR-5 or PBS (all 3×10⁷ pfu/ml). RAD001 (5 mg/kg/d) was administered by gavage; TMZ (7.5 mg/kg/5 days) was administered intraperitoneally. All animal studies were performed in the veterinary facilities of the M.D. Anderson Cancer Center in accordance with institutional, state, and federal laws and ethics guidelines for experimental animal care.

Immunohistochemical Analysis. To detect adenoviral E1A and hexon proteins in the tumor xenografts, paraffin-embedded sections of the mouse tumors were deparaffinized and rehydrated with xylene and ethanol following conventional procedures (Falkeholm, 2001). Endogenous peroxidase activity was quenched by incubating the sections in 0.3% hydrogen peroxide in methanol for 30 minutes. These sections were then treated with either goat anti-hexon antibody (diluted 1:200; Chemicon) or goat anti-E1A (diluted 1:200; Santa Cruz) at 40° C. overnight. For immnunohistochemical staining, Vectastain ABC kits (Vector Laboratories) were used according the manufacturer's instructions.

Bioluminiscence imaging. Cells were seeded at a density of 1×10⁶ cells in 100 mm dishes and cultured for 24 h. Cells were then transfected with 250 ng of E2F1 reporter plasmid (Johnson, 1994) by using FuGENE 6 transfection reagent (Roche Diagnostics Corp.). Where indicated cells were treated with pRb cDNA. Cells were harvested 48 h after treatment, and implanted in the brain of athymic mice. The mice were anesthetized 48 h later (isoflurane) and imaged for E2F-luc induced luciferase expression was performed (after i.p. injection of D-luciferin (4 and 150 g per g body weight) using the IVIS imaging system (Xenogen). Acquisition parameters were: exposure time, 5 min; binning, 4; no filter; f/stop, 1; FOV, 10 cm.

Statistical Analysis. For the in vitro experiments, statistical analyses were performed using a two-tailed Student's t test. Data are expressed as mean±SD or 95% confidence intervals (CIs). The in vivo cytopathic effect of ICOVIR-5 on human glioma xenografts was assessed by plotting survival curves according to the Kaplan-Meier method. Survival in different treatment groups was compared using the log-rank test.

Results

Structure of the oncolytic adenovirus ICOVIR-5. ICOVIR is a recombinant human adenovirus C serotype 5 which genome has been modified to encompass the following elements: 1. preceeding the E1A region: substitution of the native E1a promoter region by E2F1 responsive elements and the DM-1 insulator, and insertion of the Kozak sequence before the E1A starting ATG. 2. Within the E1A region: deletion of 24 nucleotides in the Rb-binding CR2 region. 3. Insertion of the RGD-4C peptide in the HI loop of the fiber. ICOVIR is described in detail in appication number PCT/ES03/00140, which is incorporated herein by refernce in its entiretyl

E2F-mediated E1A expression in ICOVIR-infected cells. In order to assess the transcriptional activity of the E2F1 in cancer and normal cells, U87 MG, U251 MG or arrested NHA were transfected with a E2F1-Luc reporter construct (Johnson, 1994). The E2F1 transcriptional activity was 12 and 14 folds higher in U-87 MG and U-251 MG respectively (p<0.001) than in arrested NHA where the E2F1 activity was below the level of detection. To evaluate the responsiveness of the E2F1 promoter to the virus infection, U-87 MG and U-251 MG cells transfected with E2F-Luc were infected with WT-RGD (Hong, 1999), Delta-24-RGD (Suzuki, 2001; Fueyo, 2003) or ICOVIR-5 and 24 hours later luciferase activity was measured. Glioma cells infected with any of the three adenoviruses showed a minimum increase of 10-fold in luciferase activity (p<0.001), in comparison with mock infected cells. Similar results (9.5 and 6.8 folds in cells infected with WT-RGD and Delta-24-RGD adenovirus, respectively) were obtained in growth arrested NHA. However, the infection with ICOVIR-5 of NHA did not result in a significant increase (p>0.05) in the activity of the E2F promoter (1.45-fold increase in comparison with adenovirus control). Next, the inventors examined the capability of the virus to bind and utilize the cellular E2F1 by performing a series of ChIP assays in cancer and normal cells. Thus, ChIP assays performed in U87 MG and U251 MG glioma cells 24 hours after the infection with ICOVIR-5 demonstrated the direct interaction of the E2F1 protein and the adenoviral genome. This association between E2F1 and ICOVIR was not detected when the samples were immunoprecipitated with RB suggesting the presence of “free” E2F1 protein in cancer cells. On the contrary, and as expected, in serum-starved NHA, the RB protein was recruited to the recombinant E2F-responsive elements of ICOVIR, suggesting the formation of Rb/E2F 1 repressor complex with the artificial transcription system of the adenovirus.

Cell cycle profile in ICOVIR-infected cells. Since the virus ability to induce S phase will determine its replication capability next, the inventors analyzed the cell cycle profile of U87 MG, U251 MG gliomas cell lines and arrested NHA following infection with WT-RGD, Delta-24-RGD or ICOVIR-5 adenoviruses. Flow cytometric analyses of cell cycle profiles at various times post infection showed that ICOVIR-5, 24 hours post infection, induced S phase entry to a similar extend (p<0.05) than control adenovirus (57, 43% and 35% for U87-MG and 50, 52 and 45% in U251-MG). Beyond 24 hours the cell cycle profile as measured by DNA content was profoundly disrupted and did not allow quantification of the S phase population. In contrast, in serum-starved NHA, ICOVIR-5 could not override the cell cycle arrest and induced a negligible accumulation of cells in the S phase (8%), that was significantly lower (p<0.001) than that observed in WT-RGD-(68%) and Delta-24-RGD-infected cells (35%). Interestingly, E2F1 transcriptional activity, E2F1 mRNA levels, and percentage of cells in S phase correlated with expression levels of E1A in U87 MG, U251 MG and arrested NHA astrocytes after viral infection. In these experiments, E1A expression levels in glioma cell lines were similar in samples infected with either ICOVIR-5 or adenoviral controls. Of importance, ICOVIR-infected NHA showed very low levels of E1A, while cells infected with control adenoviruses showed similar levels to those observed in cancer-infected cells.

Replication capability of ICOVIR-5 in cancer cells. Qualitative (crystal violet) and quantitative (MTT) dose-dependent assays in U-87 MG and U251 MG cells showed that ICOVIR-5 induced cytophatic effect in U-87 MG and U-251 MG glioma cells. Crystal violet staining showed that ICOVIR-5 infection resulted in cell death in both cell lines at a MOI of 1. In addition, MTT assay showed that the ICOVIR-5 LD₅₀ in both gliomas cell line ranged between 1 and 5 MOI, similar to the WT adenovirus. In order to ascertain whether the cytopathic effect was due to E1A-mediated toxicity or was sustained by an effective replication process a tissue-culture infection dose-replication assay was performed in U-87 MG and U-251 MG glioma cell line. The results showed that ICOVIR-5 replicated efficiently in both cell lines (5.0×10⁸ and 5.0×10⁹ pfu/ml respectively), very similar to the replication capacity of Delta-24-RGD (3.1×10⁹ and 1.2×10¹⁰ pfu/ml respectively). These data showed that ICOVIR-5 replicates significantly better (P<0.01) than Delta-24. The results of the replication assays were consistent with the levels of expression of early and late adenoviral genes as assessed by QT-RT-PCR and Western blot. Thus, infection with ICOVIR-5 resulted in an increase of 18.1±3.4 and 19.3±2.5 folds in E1A mRNA expression in U-87 MG and U251 MG, respectively, in comparison with mock-infected samples. These values were similar to those observed in cells infected with adenovirus control in which we observed a 12- to 27-fold increase in the level of E1A mRNA. In addition, the levels of fiber mRNA in ICOVIR-5 infected samples increased by 17±3.7 and 18±5.3 folds in U87 MG and U251 MG, respectively. These values were significantly higher (P<0.001) than those of E1A and fiber expression in Delta-24-infected cells, but similar to the observed in cells infected with the other adenovirus controls.

Effect of RB pathway restoration in ICOVIR-5 activity. The inventors next sought to determine whether restoration of pRB pathway would affect the oncolytic properties of ICOVIR-5 by performing ChIP assays in glioma cells pre-treated with exogenous Rb and then infected with ICOVIR. Thus, U87 MG and U251MG glioma cell lines were infected with an adenoviral vector carrying the exogenous wild type RB cDNA, or the Ad5CMV-pA adenovirus control (100 MOI) and 72 hours later were infected with ICOVIR-5 (10 MOI). The inventors observed that while immunoprecipitation with Rb did not show the interaction of Rb/E2F 1 complexes with the E2F responsible elements of ICOVIR in cancer cells pretreated with an empty adenoviral vector, pRb formed complexes with E2F and physically interacted with the E2F1 virus promoter in cancer cells expressing an ectopic Rb protein.

To further confirm the hypothesis that the ICOVIR-mediated cytopathic effect is dependent on the cell-cycle regulatory function of the Rb protein, U87 MG and U251 MG were infected with an adenoviral vector carrying the Rb cDNA, p21 cDNA or the Ad5CMV-pA adenovirus. 72 hours later were infected with Delta-24-RGD, ICOVIR-5 or UV-inactivated adenovirus. As expected, cell cultures pretreated with Ad5CMV-pA, were sensitive to the lytic effect of ICOVIR-5 and showed a complete cytopathic effect (91.4±1.9 and 90.2±2.3% decrease in viability in U87 MG and U251 MG respectively) within 5 days after the viral infection. In contrast, cells infected with the Ad5CMV-Rb acquired an oncolytic-resistant phenotype with 95±2.3 and 92±3.4% increase in viability in U87 MG and U251 MG respectively, that persisted for at least 10 days after infection with ICOVIR. To further confirm the virus-suppressive effect of the Rb protein, the ability of the cyclin-dependent kinase inhibitor p21, a regulator of Rb function, to reduce the effect of ICOVIR-5 on the viability of wild-type Rb cells was examined. P21-pretreatment provided almost complete protection against ICOVIR-5 as reflected by 90.1±2.1 and 87.4±3.8% increase in viability in U87 MG and U251 MG respectively. Importantly, the Rb-suppressive and p-21-suppressive effects were higher in cells infected with ICOVIR than in cells infected with Delta-24-RGD. Thus, the rescue of the viability was observed in approximately 50% of cells infected with Delta-24-RGD. The difference in the sensitivity of ICOVIR and Delta-24-RGD to the function of negative regulators of cell cycle evidenced the effect that the transcriptional regulation of E1A in ICOVIR added to the modification of the interaction E1A/Rb present in both ICOVIR and Delta-24-RGD. To correlate cell death and virus replication, plaque forming assays were performed that showed a dramatically decrease in the replication capability of ICOVIR in cells overexpressing Rb or p21. To ascertain the mechanism of the Rb-mediated suppression of ICOVIR replication, QT-RT-PCR was used to analyze the amount of E1A and fiber transcripts in ICOVIR-infected cells. In cultures pretreated with Rb the inventors detected very low levels of E1A mRNA transcripts (1.5±1 and 2.1±1.2 folds in U-87 MG and U251 MG respectively). In addition, fiber expression was below the level of detection in these cultures. These results were similar in cultures pre-treated with p21, thus indicating the defective expression of E1A and the impaired replication ability of the vector upon restoration of a functional RB pathway. These results further confirm the higher dependence of ICOVIR-5 replication capability in the RB function and in addition, showed that the combination of redundant controls targeting the RB pathway results in improved tumor specificity.

Therapeutic index of ICOVIR-5. Because Rb function is one of the major differences between normal and glioma cells, the effect of ICOVIR-5 infection in growth-arrested NHA were examined. Three days after serum starvation NHA were infected with ICOVIR-5 at doses of 0.1 to 10 MOI. Cytotoxicity was evaluated 7 days post-infection by MTT assay. The experiments revealed that at the maximum dosage used (10 MOI) ICOVIR-5 elicited 20±5.2% cytotoxitcity, a result underscored by the fact that the LD₅₀ of Delta-24-RGD and WT was 1 MOI. To determine the therapeutic index (i.e., viral replication in tumor cells/viral replication in normal cells), the inventors compared the replication of this adenovirus in serum-starved gliomas cells and in NHA. Under these conditions, ICOVIR-5 showed a drastic reduction of the replication capability in NHA. As expected, Delta-24-RGD displayed an attenuated replication phenotype. In contrast WT and WT-RGD adenoviruses replicated with the same efficiency in both gliomas and NHA. Expression of E1A mRNA and protein levels were significantly reduced (2±0.3 folds) in arrested NHA treated with ICOVIR-5 in comparison with adenovirus control WT (14.4±3.3), WT-RGD (17.5±5), Delta-24 (5±1.4) and D24-RGD (8.5±2.9). Confirming impaired replication capability, fiber expression levels were absent at both levels (mRNA and protein) in ICOVIR-5 infected samples.

Transcritional activity of E2F1 in human glioma xenografts infected with ICOVIR. To examine the transcriptional activity of E2F1 within a glioma xenograft in vivo, U-87 MG cells transfected with the E2F-Luc construct were implanted in the brain of nude mice followed, to confirm the presence of the tumor, by MRI imaging of the brain. It was shown for the first time that U-87 MG xenografts expressed high luciferase activity (1.6×10⁵ light units, l.u.). Infection of the E2F-luc-transfected U-87 MG with ICOVIR-5 elicited increased transcriptional activity of the E2F promoter resulting in higher luciferase activity (1.4×10⁶ l.u). Testing again the capability of Rb to repress E2F transcriptional activity, now in vivo, E2F-Luc-transfected U-87-MG cells treated with an exogenous pRb were injected intracranially. The overexpression of Rb resulted in a decreased E2F1-promoter activity (6.3×10⁴ lU). Of importance, infection with ICOVIR-5 in Rb-pretreated cells did not increase E2F1 transcriptional activity as measured by luciferase activity (5×10⁴ l.u). Next, the inventors assessed whether the free cellular E2F1 could physically interact with the ICOVIR-5 with the recombinant E2F-responsive elements in vivo. Three days after implantation, U-87 MG xenografts were intratumorally infected with ICOVIR, and then 35 days later brains were collected and subjected to ChIP assay. The inventors were able to detect the interaction between the free E2F1 in U87 MG xenografts and the E2F-promoter sequence encompassed in the ICOVIR genome. To the best of our knowledge these data represent the first demonstration of the ability of an oncolytic adenovirus to enhance E2F transcriptional activity and to physically interact with E2F1 protein in vivo within an experimental glioma.

ICOVIR-mediated Toxicity. Although the use of oncolytic adenoviruses as glioma therapy has been confined to intracranial injections, it is probably impossible to avoid extravasation of the adenovirus to the blood stream, and for that reason assessment of toxicity after systemic delivery may be considered as a pre-requisite before clinical testing. To evaluate the specificity and thus possible toxicity, ICOVIR-5 mediated toxicity was assessed after a single intravenous or intracarotid injection. Weight loss, overall survival, liver enzymes (AST and ALT) and hematological profile were determined at day 5 post-injection. Whereas an intravenous dose of 5×10¹⁰ viral particles (vp) was LD₅₀ for WT or Delta-24-RGD-injected mice, the LD₅₀ of ICOVIR-5 was 1×10¹¹ vp. At a dose of 5×10¹⁰ vp, ICOVIR-5 administration only induced a moderate increase in transaminase levels but no other signs of general toxicity such as reduction in body weight were observed. Moreover, and in contrast to the significant reduction in platelets and lymphocytes associated to a single intravenous injection of 5×10¹⁰ vp of WT, no hematological alterations were detected after the administration of ICOVIR-5 at a similar (5×10¹⁰ vp ) or higher dose (1×10¹¹ vp). Intriguingly, intracarotid delivery of ICOVIR-5 at dose of 1.5×10¹³ vp/animal did not negatively affected survival and the serum levels of transaminases remained within normal values (data not shown).

The inventors also examined the expression of E1A protein by immunostaining of the liver. It was shown that, the expression of E1A was efficiently restricted in ICOVIR-5-injected mice even at the highest intravenous dose (1×10¹¹ vp), compared to samples from mice injected with WT or Delta-24-RGD at 5×10¹⁰ vp. A similar analysis was carried out after intracarotid injection, and E1A protein levels were determined in liver, lung and brain samples and the expression of E1A was significantly attenuated in the animals treated with ICOVIR-5 versus mice treated with control adenovirus (data not shown). Moreover, histological analysis of liver samples obtained at day 3 after systemic administration (FIG. 6G) showed marked differences in mice treated with ICOVIR or any of the adenovirus controls. Thus, a single 5×10¹⁰ vp dose of WT or Delta-24-RGD resulted in signs of hepatitis (macrosteatosis, presence of Councilman bodies and large necrotic areas). In contrast, livers from mice treated with ICOVIR-5 at the same dose displayed a practically normal phenotype, with only marginal Councilman bodies in the more superficial areas. The analysis of ICOVIR-5-injected livers at later time points (day 5) demonstrated the presence of mitotic nuclei and reduced necrotic areas, which is suggestive of a regeneration process (data not shown).

ICOVIR-5 antiglioma efficacy in vivo. To test the in vivo therapeutic effect of ICOVIR-5 in vivo, U87 MG xenografts were grown in the brain of athymic mice. The animals received three intratumoral injections (3×10⁸ pfu, day 3, 5 and 7 post implantation) of PBS, ICOVIR-5 or control adenovirus. The mean survival for the control mice (i.e., mice receiving PBS and UV-inactivated ICOVIR-5) was 31.5 days. All the mice treated with PBS or UVi died by day 33 and there were no long-term survivors. In contrast, mice treated with ICOVIR-5 yielded 28.6% of long-term survivor. Examination of the brains of ICOVIR-5 treated mice that died between 20 and 65 days after treatment indicated that their deaths resulted from the mass effect of large ellipsoid tumors. Further examination of these brains at higher magnification revealed the presence of prominent viral inclusions. Immunohistochemical staining for E1A protein revealed three distinct and concentric tumor zones: inner most central core of necrosis and cellular debris; a middle zone with high E1A protein expression, which consists of large numbers of tumor cells with prominent viral inclusions intermixed with apparently intact tumor cells; and a peripheral zone of intact tumor cells with intact tumor cells with few scattered cells with signs of infection. Importantly, immunohistochemical staining for hexon demonstrated the ability of the virus to transcribed and translate late genes thus indicating the replication capability of ICOVIR-5. Immunohistochemical analyses of normal regions of brain tissue in animals treated with ICOVIR-5 were negative for E1A and hexon viral proteins (data not shown). Expression of early and late genes was also detected in the tumor, but not in the normal brain with QT-RT-PCR. Examination of the asymptomatic long-term survivors brains showed complete tumor regression in all the animals but one in which the persistence of a small tumor was detected (Data not shown). In the mice without tumors some tumors sequelae were observed, including dystrophic calcification and microcyst formation. Immunohistochemical analyses of the brains of the long-term survivors with both anti E1A and anti-hexon antibodies did not reveal persistence of the adenovirus (data not shown). The inventors did not observe either E1A expression or signs of inflammatory reaction in the normal brain in any of the examined mice.

Combination treatment with ICOVIR-5 and temozolomide or RAD001. Antitumor efficacy in combination with chemotherapy was evaluated in the U87-MG tumor model using intratumoral injection of ICOVIR-5 and oral administration of Temozolamide (TMZ) or RAD001 First, the cytotoxic potential of these drugs alone or in combination with ICOVIR-5 were assessed in vitro in the U-87 MG cell line. The combined administration of any of the two drugs with ICOVIR-5 enhanced the antitumor properties of the virus reducing the dosage needed to achieve the LD₅₀. Adenoviral fiber expression analyses by western blot and RT-QT-PCR showed that neither RAD001 nor TMZ suppressed adenoviral replication in vitro. To analyze the in vivo therapeutic effect of ICOVIR-5 in combination with RAD001 and TMZ, U87-MG xenografts were grown in the brain of athymic mice. The animals received three intratumoral injections (day 3, 5 and 7 post implantation) of PBS or ICOVIR-5 (3×10⁷ pfu/mouse) and/or RAD001 (5 mg/kg/d) or TMZ (7.5 mg/kg/5 days). The mean survival for the control mice (PBS or UV-inactivated ICOVIR-5) was 30.5 days. In addition, the main survival for the animals treated with the drugs or the ICOVIR-5 alone were RAD001 49 days, TMZ 35.5 days and ICOVIR-5 35 days. In contrast, animals treated with the combination virus and drugs showed a significantly increased in the mean survival ICOVIR-5 plus RAD001 72.5 days and ICOVIR-5 plus TMZ 62 days, both of the combination treatment were significant as assessed by the Log-Rank test (P<0.0001). Importantly, ICOVIR-5 in combination with RAD001 or TMZ led to 40 and 50% of long-term survival animals. Examination of the brains in animals treated with RAD001 or TMZ alone revealed a large area of necrosis in the center of the tumor and scattered smaller ones. Immunohistochemical staining for E1A and hexon protein in the brain of mice treated with either ICOVIR-5 alone or the drug combination showed staining for both indicating the capability of the virus not only to infect but to replicate efficiently even in the presence of the drugs. These data were further confirmed by quantification of mRNA expression levels of E1A and fiber. These analyses showed very similar mRNA levels of E1A and fiber in tumor tissue treated with ICOVIR-5 alone or in combination with RAD001 or TMZ.

One of skill in the art readily appreciates that the present invention is well adapted to carry out the objectives and obtain the ends and advantages mentioned as well as those inherent therein. Methods, procedures and techniques described herein are presently representative of the preferred embodiments and are intended to be exemplary and are not intended as limitations of the scope. Changes therein and other uses will occur to those skilled in the art which are encompassed within the spirit of the invention or defined by the scope of the pending claims.

### REFERENCES

The following references, to the extent that they provide exemplary procedural or other details supplementary to those set forth herein, are specifically incorporated herein by reference.
U.S. Patent Appln. 20030138405
Campa et al, Biochem. Biophys. Res. Commun., 275(2):631-636, 2000.
Chow and Pearson, Proc. Natl. Acad. Sci. USA, 82:2247:2251, 1985.
D'Arpa et al., Cancer Res., 50:6919:6924, 1990.
Ekstrand et al, Proc. Natl. Acad. Sci. USA, 89(10):4309-4313, 1992.
Flint and Shenk, Annu. Rev. Genet., 31:177:212, 1997.
Frederick et al, Neuro-oncol., 2(3):159-163, 2000.
Fueyo et al., J. Natl. Cancer Inst., 95:652:660, 2003.
Fueyo et al., Nature Medicine, 4(6):685-690, 1998.
Fueyo et al, Oncogene, 19:2-12, 2000.
Geoerger et al., Cancer Res., 62(3):764-772, 2002.
Ghosh and Bachhawat, In: Liver Diseases, Targeted Diagnosis and Therapy Using Specific Receptors and Ligands, Wu et al. (Eds.), Marcel Dekker, NY, 87-104, 1991.
Gomez-Manzano et al., Neurology, 63:418:426, 2004.
Hess et al., Neuro-Oncol., 1(4):282-288, 1999.
Jacotot, Acad. Sci. Hebd. Seances Acad. Sci., 264(22):2602-2603, 1967.
Jones and Shenk, Cell, 17:683:689, 1979.
Kim, Expert Opin. Biol. Ther., 1(3):525-538, 2001.
Kuan et al., Endocr. Relat. Cancer, 8(2):83-96, 2001.
Lal et al, J. Neurosurg., 92:326:333, 2000.
Liu et al., Proc. Natl. Acad. Sci. USA, 78:3487:3491, 1981.
Lorimer et al, Proc. Natl. Acad. Sci. USA, 93(25):14815-14820, 1996.
PCT Appln. WO 96/12406
Remington's Pharmaceutical Sciences, 15^{th} ed., 33:624-652, Mack Publishing Company, Easton, Pa., 1980.
Romig and Richter, Nucleic Acids Res., 18:801:808, 1990.
Schiffer, Forum, 8(3):244-255, 1998.
Shapiro and Shapiro, Oncology, 12(2):233-240, 1998.
Sugawa et al. Proc. Natl. Acad. Sci. USA, 87(21):8602-5606, 1990.
Sugimoto et al, Cancer Res., 50:6925:6930, 1990.
Suzuki et al., Clin. Cancer Res., 7:120-126, 2001.
Trask and Mulle, Nucleic Acids Res., 11:2779:2800, 1983.
Vile, Cancer Gene Ther., 9(12):1062-1067, 2002.
Wildner et al., Cancer Res., 59:410-413, 1999.
Yan et al., J. Virol., 77(4):2640-2650, 2003.

What is claimed is:

1. A method of treating a patient with cancer comprising:
a) administering to the patient an effective amount of a cell cycle modulating agent that elevates the proportion of cancer cells in S-phase of the cell cycle; and
b) administering an effective amount of a second anti-cancer therapy to a subject in need thereof.

2. The method of claim 1, wherein the cell cycle modulating agent is administered to the subject before administration of the anti-cancer therapy.

3. The method of claim 1, wherein the cell cycle modulating agent is administered to the subject at the same time as administration of the anti-cancer therapy.

4. The method of claim 1, wherein the cell cycle modulating agent is administered to the subject after administration of the anti-cancer therapy.

5. The method of claim 1, wherein the cell cycle modulating agent is a virus, small molecule, peptide.

6. The method of claim 5, wherein the virus is an adenovirus.

7. The method of claim 6, wherein the adenovirus is an oncolytic adenovirus.

8. The method of claim 7, wherein the oncolytic adenovirus is a Delta 24 adenovirus.

9. The method of claim 6, wherein the oncolytic adenovirus comprises a targeting moiety.

10. The method of claim 9, wherein the targeting moiety comprises a modified fiber protein.

11. The method of claim 10, wherein the modified fiber protein is modified by an insertion of heterologous amino acids in the fiber protein.

12. The method of claim 11, wherein the heterologous amino acid sequence is inserted in the HI loop of the fiber protein.

13. The method of claim 11, wherein the heterologous amino acid sequence is a RGD, amino acid sequence.

14. The method of claim 9, wherein the oncolytic adenovirus has a decreased E1A mediated toxicity.

15. The method of claim 14, wherein the E1A mediated toxicity is reduced by modulation of E1A expression.

16. The method of claim 15, wherein modulation of E1A expression is effected by substitution of the E IA promoter with a heterologous promoter.

17. The method of claim 16, wherein the heterologous promoter is two E2F1 promoter sequences.

18. The method of claim 17, wherein the two E2F1 promoter sequences are preceded by an insulator sequence.

19. The method of claim 1, wherein the anti-cancer therapy is radiation therapy, chemotherapy, immunotherapy, gene therapy, or anti-angiogenic therapy.

20. The method of claim 19, wherein the anti-cancer therapy is chemotherapy.

21. The method of claim 20, wherein the chemotherapy is at the S-phase of the cell cycle.

22. The method of claim 21, wherein the chemotherapy is an antimetabolite.

23. The method of claim 21, wherein the chemotherapy is a topoisomerase I inhibitor.

24. The method of claim 23, wherein the topoisomerse I inhibitor is CPT11.

25. The method of claim 21, wherein the chemotherapy is CPT-11, temozolomide, or a platin compound.

26. The method of claim 20, wherein the chemotherapy comprises an alkylating agent, mitotic inhibitor, antibiotic, or antimetabolite.

27. The method of claim 20, wherein the chemotherapy is temozolomide, epothilones, melphalan, carmustine, busulfan, lomustine, cyclophosphamide, dacarbazine, polifeprosan, ifosfamide, chlorambucil, mechlorethamine, busulfan, cyclophosphamide, carboplatin, cisplatin, thiotepa, capecitabine, streptozocin, bicalutamide, flutamide, nilutamide, leuprolide acetate, doxorubicin hydrochloride, bleomycin sulfate, daunorubicin hydrochloride, dactinomycin, liposomal daunorubicin citrate, liposomal doxorubicin hydrochloride, epirubicin hydrochloride, idarubicin hydrochloride, mitomycin, doxorubicin, valrubicin, anastrozole, toremifene citrate, cytarabine, fluorouracil, fludarabine, floxuridine, interferon α-2b, plicamycin, mercaptopurine, methotrexate, interferon α-2a, medroxyprogersterone acetate, estramustine phosphate sodium, estradiol, leuprolide acetate, megestrol acetate, octreotide acetate, deithylstilbestrol diphosphate, testolactone, goserelin acetate, etoposide phosphate, vincristine sulfate, etoposide, vinblastine, etoposide, vincristine sulfate, teniposide, trastuzumab, gemtuzumab ozogamicin, rituximab, exemestane, irinotecan hydrocholride, asparaginase, gemcitabine hydrochloride, altretamine, topotecan hydrochloride, hydroxyurea, cladribine, mitotane, procarbazine hydrochloride, vinorelbine tartrate, pentrostatin sodium, mitoxantrone, pegaspargase, denileukin diftitix, altretinoin, porfimer, bexarotene, paclitaxel, docetaxel, arsenic trioxide, or tretinoin.

28. The method of claim 27, wherein the chemotherapy comprises CPT-11, temozolomide, or a platin compound.

29. The method of claim 19, wherein radiation therapy comprises X-ray irradiation, UV-irradiation, γ-irradiation, or microwaves.

30. The method of claim 1, further comprising subjecting the subject to surgical therapy.

31. The method of claim 1, wherein the cell cycle modulating agent, the anti-cancer therapy, or both the cell cycle modulating agent and the anti-cancer therapy are adminstered intravenously, intratumorally, or intracranially.

32. The method of claim 31, wherein the cell cycle modulating agent, the anti-cancer therapy, or both the cell cycle modulating agent and the anti-cancer therapy are administered intracranially.

33. The method of claim 31, wherein the cell cycle modulating agent, the anti-cancer therapy, or both the cell cycle modulating agent and the anti-cancer therapy are administered intratumorally.

34. The method of claim 1, wherein the cell cycle modulating agent, the anti-cancer therapy, or both the cell cycle modulating agent and the anti-cancer therapy are directly injected into a tumor.

35. The method of claim 1, wherein the administration of the cell cycle modulating agent, the anti-cancer therapy, or both the cell cycle modulating agent and the anti-cancer therapy occurs more than once, twice, three, four times or more.

36. The method of claim 35, wherein the cell cycle modulating agent, the anti-cancer therapy, or both the cell cycle modulating agent and the anti-cancer therapy are administered at least three times to the patient.

37. The method of claim 31, wherein the cancer is a astrocytoma, oligodendroglioma, anaplastic glioma, glioblastoma, ependymoma, meningioma, pineal region tumor, choroid plexus tumor, neuroepithelial tumor, embryonal tumor, peripheral neuroblastic tumor, tumor of cranial nerves, tumor of the hemopoietic system, germ cell tumors, or tumor of the sellar region.

38. The method of claim 37, wherein the cancer is glioblastoma.

39. The method of claim 7, wherein from about 10³ to about 10¹⁵ viral particles are administered to the subject.

40. The method of claim 39, wherein from about 10⁵ to about 10¹² viral particles are administered to the subject.

41. The method of claim 39, wherein from about 10⁷ to about 10¹⁰ viral particles are administered to the patient.

42. The method of claim 1, further comprising determining the proportion of cells in S-phase.

43. The method of claim 42, wherein at least 40, 45, 50, 55, 60, 65, 70, 75, 80, 85, 90, or 95% of cells in a biopsy sample are in S-phase.
